(19) United States
(12) Patent Application Publication
(10) Pub. No.: US 2025/0002581 A1
(43) Pub. Date: Jan. 2, 2025

(54) HUMANIZED MONOCLONAL ANTIBODY FOR RESTORING DYSFUNCTIONAL HUMAN T AND B CELLS AGAINST CANCER AND VIRAL INFECTION

(71) Applicant: Versitech Limited, Hong Kong (CN)

(72) Inventors: Zhiwei Chen, Hong Kong (CN); Dongyan Zhou, Hong Kong (CN); Li Liu, Hong Kong (CN)

(21) Appl. No.: 18/710,523

(22) PCT Filed: Nov. 15, 2022
(86) PCT No.: PCT/CN2022/132000

§ 371 Date: May 15, 2024

Related U.S. Application Data
(60) Provisional application No. 63/264,061, filed on Nov. 15, 2021.

(51) Int. Cl. C07K 16/28; A61K 39/00; A61P 37/04

(52) U.S. Cl. CPC C07K 16/2818; A61P 37/04; A61K 2039/505; C07K 2317/24; C07K 2317/33; C07K 2317/94

(57) ABSTRACT

Provided are two fully humanized Δ42PD1-specific mAbs, specifically huCH101 and huCH34 at 97.0% humanness by CDR grafting. Provided is a method of using huCH101 and/or huCH34 to treat subjects in need of Δ42PD1-specific mAb, including subjects with cancer and/or infection.

### BACKGROUND OF THE INVENTION

Programmed death-1 (PD-1, CD279) is a member of the CD28 superfamily that negatively regulates the function of T cells through interaction with its two native ligands PD-L1 (CD274) and PD-L2 (CD273). PD-1 is a type I transmembrane receptor protein composed of a single immunoglobulin (Ig) variable-like domain, a cytoplasmic domain, and two tyrosine-based signaling motifs. PD-1 is constitutively expressed at low levels on resting T cells and is up-regulated on T cells, natural killer T (NKT) cells, B cells and macrophages upon activation.

The absence of PD-1 in some mouse models provides significant resistance against bacterial infection through innate immunity, demonstrating the importance of the regulatory role of PD-1 against pathogenic infections. In addition, PD-1 plays significant roles in a number of autoimmune diseases, including systemic lupus erythematosus (SLE) and rheumatoid arthritis.

Immune checkpoint inhibitors, such as humanized programmed cell death 1 (PD-1)-specific mAbs, have been extensively used for cancer immunotherapy. Besides PD-1, one of alternatively spliced isoforms of PD-1, namely Δ42PD1, has been discovered. Moreover, a Δ42PD1-specific mAbs, CH101 and CH34, were produced using the hybridoma technology. The immune modulation effect of Δ42PD1 has also been demonstrated, which implies that Δ42PD1 is a potential target for developing therapeutic antibodies for treating human diseases. Many cancer patients do not respond to anti-PD1 immunotherapy.

Therefore, there remains a need for an effective anti-PD1 immunotherapy, particularly for use in humans.

### BRIEF SUMMARY OF THE INVENTION

The subject invention pertains to antibodies that bind specifically to the PD-1 protein isoform Δ42PD1. In some embodiments, the antibody is a monoclonal antibody. In certain embodiments, the subject inventions are fully humanized CH101 and CH34 antibodies, namely huCH101 and huCH34, at about 97.0% humanness by CDR grafting. We also determined their specificity, binding affinity, and sensitivity for interaction with Δ42PD1.

Another aspect of the present invention provides methods for the prevention, treatment, or amelioration of cancer and viral infection. The method comprises administering to a subject in need of such prevention and treatment an effective amount of an antibody that targets PD1 protein isoform of the present invention (such as Δ42PD1 protein), a nucleic acid molecule encoding the antibody (e.g., plasmid) that targets a protein of the present invention (such as Δ42PD1 protein). In certain embodiments, huCH101 and huCH34 displays therapeutic efficacies as antibody drugs against hepatocellular carcinoma.

### BRIEF DESCRIPTION OF THE DRAWINGS

FIG. 1. Strategy for CH101 humanization via CDR grafting.

FIGS. 2A-2B. Amino acid sequences alignment for comparing the variable regions of murine CH101, germline sequence and humanized CH101 after CDR grafting. The amino acid sequences of (FIG. 2A) variable kappa (VK) and (FIG. 2B) heavy chain (VH) of CH101 or germlines were displayed. Amino acids of framework regions were colored black and CDR regions were colored red. The discrepant amino acids in the framework regions of the germline sequences were colored green whereas the variants in the framework plus CDR areas of parent CH101 were underlined.

FIGS. 3A-3I. FACS staining of Δ42PD1 by a panel of humanized CH101 antibodies. A serial of humanized CH101 antibodies were used to differentiate Δ42PD1 and PD1 on 293T cell surface by flow cytometry. The activities of distinct antibody binding were color-coded in light blue for 293T-Δ42PD1 cells, orange for 293T-PD1 cells and red for 293T cells, respectively. The parental murine CH101 was used as a positive control for 293T-Δ42PD1 binding. Human IgG1 isotype was used as a negative control. Anti-human CD279 (PD-1)-APC was used as a positive control for 293T-PD1 binding.

FIGS. 4A-4D. Evaluation of the half-life of humanized CH101 antibodies in vivo. (FIG. 4A) Animal schedule for assessment of antibody half-life. The in vivo kinetic of CH101-huIgG1 (FIG. 4B), CH101-IgG4 (FIG. 4C) and hufrCH101-IgG1 (FIG. 4D) was measured by ELISA and analyzed. The half-life of individual antibody was displayed accordingly.

FIGS. 5A-5G. The in vivo assessment of CH101-huIgG1 (chimeric) for immunotherapeutic effects in the HCC mouse model. (FIG. 5A) Experiment schedule. (FIG. 5B) The Huh7 tumor of each animal at the endpoint. (FIG. 5C) The tumor growth overtime was documented. (FIG. 5D) Tumor weight of each animal at the endpoint was measured and compared between groups. (FIGS. 5E-5G) The profile of inflammatory cytokine in the plasma at the endpoint were measured (except the samples of two animals in CH101-huIgG1 group and one animal in murine CH101 group unavailable for assessment). Ordinary one-way ANOVA besides multiple comparisons test were used for statistics analysis. *p<0.05; **p<0.01, ***p<0.001.

FIGS. 6A-6G. The in vivo assessment of hufrCH101-huIgG1 for immunotherapeutic effects in the HCC mouse model. (FIG. 6A) Experiment schedule. (FIG. 6B) Images of Huh7 tumor collected from each animal at the endpoint. (FIG. 6C) The tumor growth overtime was documented weekly. (FIG. 6D) Tumor weight of each animal at the endpoint was measured. (FIGS. 6E-6G) The profile of inflammatory cytokine in the plasma at the endpoint were measured Ordinary one-way ANOVA besides multiple comparisons test were used for statistics analysis. *p<0.05; **p<0.01, ***p<0.001.

FIGS. 7A-7I. High levels of Δ42PD-1 expression on distinct T cell subsets in HCC patients. (FIGS. 7A-7B) Representative dot plots and quantified results (means±SEM) of Δ42PD-1⁺ and PD-1⁺ T cells in PBMCs of 38 HCC patients and 43 healthy people. (FIGS. 7C-7D) Representative dot plots and quantified results (means±SEM) of Δ42PD-1⁺CD4⁺ and CD8⁺ T cells in PBMCs of these HCC patients. (FIGS. 7E-7F) Representative dot plots and quantified results (means±SEM) of Δ42PD-1⁺ or PD-1⁺ T cells in paired tumor and adjacent non-tumor tissues derived from 25 HCC patients. (FIG. 7G) Frequencies of Δ42PD-1⁺CD4⁺ or Δ42PD-1⁺CD8⁺ T cells in paired tumor and adjacent non-tumor tissue from the same patients (n=25). (FIG. 7H) Correlations between frequencies of Δ42PD-1⁺ and PD-1⁺ T cells in PBMCs (n=38) and tumors (n=25) respectively in the same cohort of HCC patients. (FIG. 7I) Frequencies (means±SEM) of PD-1⁺ and Δ42PD-1⁺ T cells in PBMCs and tumors of HCC patients stratified into their UICC8 staging group (PBMCs: stage I (n=15), stage II (n=11) and III (n=9); tumors: stage I (n=7), stage II (n=9) and III (n=7)). p<0.05 was considered significant: * p<0.05, ** p<0.01 and *** p<0.001.

FIGS. 8A-8N. Anti-Δ42PD-1 antibody inhibits HCC development in murine models. (FIG. 8A) Growth curves of Huh7 and PLC5 in NSG-huPBL mice (n=6/group). (FIGS. 8B-8C) Representative dot plots and frequencies of PD-1⁺Δ42PD1⁻ and PD-1⁺Δ42PD-1⁺ T cells out of total human CD45⁺CD3⁺ T cells in spleen or in TME were determined in NSG-huPBL and Huh7-NSG-huPBL mice. (FIG. 8D) Schematic representation of treatment schedule. Huh7 cells were s.c. inoculated and left for growth for 1 week before receiving daily intraperitoneal injection of 5 doses of 200 μg CH101, anti-PD-1 antibody (Nivolumab) or isotype, respectively. (FIG. 8E) Tumor growth curve of Huh7-NSG-HuPBL mice after CH101, anti-PD-1 Nivolumab or isotype treatment. Results shown are representative of two independent experiments with consistent findings (n=4/group). (FIG. 8F) Tumor growth curves (means±SEM) were determined for Huh7 in NSG mice or Huh7-TLR4KO in NSG-HuPBL mice (n=4/group). (FIGS. 8G-8H) Growth of orthotopic MHCC-97L tumors was measured by bioluminescent imaging and was expressed as the mean S/S0 value (H), where S is the bioluminescence signal on the day of evaluation and S0 is the bioluminescence signal before treatment (n=9/group). Results were cumulative data from 2 independent experiments. (FIG. 8I) One representative liver image is shown at the endpoint. Arrows indicate intrahepatic tumors. (FIG. 8J) Spearman's correlations of Δ42PD-1⁺ T cells and IL-6 intensity in HCC patients. The dashed line indicated linear regression. (FIG. 8K) Representative images of one patient's tumor sample (×40) indicated the co-localization of Δ42PD-1⁺ TIL with IL-6-producing TLR4⁺ HCC cells. (FIG. 8L) Quantification (means±SEM) of Δ42PD-1⁺ and PD-1⁺ T cells in PBMCs, spleens and tumors of Huh7-NSG-huPBL mice at the endpoint. (FIG. 8M) Quantification (means±SEM) of IL-6 and IL-8 in plasma at the endpoint. (FIG. 8N) Representative immunofluorescent staining (×20) is shown for tumor tissues derived from Huh7-NSG-huPBL mice, together with colour-coding for cell expression of IL-6, TLR4 and CD3, respectively. p<0.05 was considered significant: * p<0.05, ** p<0.01 and *** p<0.001.

FIG. 9. Light and heavy chain sequences of the murine Δ42PD-1-specific CH34 monoclonal antibody and the color-coded CDR regions essential for making humanized monoclonal antibodies.

FIG. 10. Light chain variable region (VL) and heavy chain variable region (VH) sequences of the humanized Δ42PD-1-specific CH34 monoclonal antibody and the color-coded CDR regions essential for making humanized monoclonal antibodies.

FIG. 11. Specificity of cloned (top) and hybridoma-derived anti-Δ42PD1 antibody CH34 for binding to Δ42PD1 but not to PD-1 expressed on human 293T cells.

FIG. 12. Specificity of cloned anti-Δ42PD1 antibody CH34 for binding to Δ42PD1 but not to PD-1 expressed on human B cells.

FIG. 13. Increased Δ42PD-1 expression accompanies B cell exhaustion in CHPs. (FIG. 13A) Representative flow cytometry analysis of CD19⁺ B cells derived from BDs (n=14) and untreated CHPs (n=42). Numbers represent percentages of gated populations. (FIG. 13B) Summary of data from FIG. 13A showing Median Fluorescence Intensity (MFI) of Δ42PD-1 and frequencies of Δ42PD-1⁺ B cells in BDs and CHPs. (FIG. 13C) Color-coded gating on CD21/CD27-stained B cells of a representative BD and CHP to identify CD21⁻CD27⁺ activated memory (AM), CD21⁻CD27⁺ resting memory (RM), CD27⁻CD21⁻ tissue like memory and anergic naïve B (TLM/aN), and CD27⁻CD21⁺ resting naïve plus intermediated memory B cells (IM/rN). (FIG. 13D) Summary of data from FIG. 13C showing increased frequencies of AM and TLM/aN B cells in CHPs in comparison with BDs. (FIG. 13E) Δ42PD-1 expression on color-coded AM, TLM/aN, RM and IM/rN B cells of a representative BD and CHP. Numbers indicate MFI of Δ42PD-1 on cells. (FIG. 13F) Summary of data from FIG. 13E showing MFI of Δ42PD-1 on B cell subsets in BDs and CHPs. (FIG. 13G) Representative flow cytometry analysis of PD-1⁺ and Δ42PD-1⁺ AM, TLM/aN, RM and IM/rN B cells derived from BD and CHP. (FIG. 13H) Summary of data from FIG. 13G showing frequencies of Δ42PD-1⁺ AM, TLM/aN, RM and IM/rN B cells in BDs and CHPs. Data represent one measurement of each sample. Each dot in FIG. 13B, FIG. 13D, FIG. 13F and FIG. 13H represents a single individual. Statistical significance is determined using ordinary one-way ANOVA and the two-stage linear step-up procedure of Benjamini, Krieger and Yekutieli, with a single pooled variance for FIG. 13B, unpaired t-test for FIG. 13D, and ordinary two-way ANOVA and the two-stage linear step-up procedure of Benjamini, Krieger and Yekutieli, with a single pooled variance for FIG. 13F and FIG. 13H. *, P≤0.05; **, P≤0.01; ***, P≤0.001 and ****, P≤0.0001. Averaged data are presented as the mean±SEM.

FIG. 14. Anti-Δ42PD-1 antibody reduces SHP1 recruitment, enhances AKT1/FOXO1 pathway and B cell proliferation. (FIG. 14 A) Representative flow cytometry analysis. (FIG. 14B) Summary of data from FIG. 14A. Primary B cells from BDs (n=6) are prelabelled with FITC-CFSE and stimulated with α-IgM/G F(ab′)2 (10 μg/ml) plus msIgG2b control antibody or CH34. Cells are analyzed by flow cytometry at day 5. (FIG. 14C) CH34 reduces SHP1 recruitment by Δ42PD-1. Primary B cells from BDs are stimulated with α-IgM/G F(ab′)2 (10 μg/ml) plus msIgG2b control antibody or CH34, and lysed at day 2 for immunoprecipitation and western blot using antibodies against Δ42PD-1 and SHP1, respectively. (FIG. 14D) CH34 treatment expanded Ki67⁺ B cell population. (FIG. 14E) CH34 treatment increases phoph-AKT1-S473 and phosph-FOXO1-S256 level, and decreases P27 protein expression. (FIG. 14F) CH34 treatment increases FOXO1 and P27 mRNA in B cells (n=3). In FIGS. 14D-F, Primary B cell are cultured in complete RPMI1640 medium with α-IgM/G F(ab′)2 (10 μg/ml) and msIgG2b control antibody (10 g/ml) or CH34 (10 μg/ml), or SHP1 inhibitor NSC87877(10 uM). 48 h later, cells are stained for flow cytometry analysis (FIG. 14D) or lysed for western blotting detection of AKT1, FOXO1 and P27 (FIG. 14E), or qPCR for mRNA of FOXO1 and P27 (FIG. 14F). Statistical significance is determined using paired t-test. *, p<0.05 and ***, p<0.001. Averaged data are presented as the mean±SEM.

FIG. 15. CH34 enhances B cell proliferation after BCR-stimulation in HIV patients. (FIGS. 15A-B) CH34 increases proliferative response of naïve (IgD⁺) and memory (IgD⁻) B cell after BCR-stimulation (n=20). (FIG. 15A) Representative flow cytometry analysis. (FIG. 15B) Summary of data from FIG. 15A. (FIGS. 15C-D) CH34 increases CD86 expression in naïve and memory B cellspf CHPs upon BCR stimulation (n=20). (FIG. 15C) Representative flow cytometry analysis. (FIG. 15D) Summary of data from FIG. 15C. (FIGS. 15E-F) CH34 increases proliferative response of B cells from CHPs after TD stimulation (n=10). (FIG. 15E) Representative flow cytometry analysis. (FIG. 15F) Summary of data from FIG. 15E. (FIGS. 15G-H) CH34 increases proliferative response of naïve B cells from CHPs after TI stimulation (n=10). (FIG. 15G) Representative flow cytometry analysis. (FIG. 15H) Summary of data from FIG. 15G. IgD⁺ and IgD⁻ B cells isolated from PBMC of CHPs are prelabelled with CFSE and cultured in RPMI1640 medium with α-IgM/G F(ab′)2 (10 μg/ml), TD or TI stimulation plus CH34 (10 μg/ml) or msIgG2b control (10 g/ml) for 5 days, and analyzed by flow cytometry. Statistical significance is determined using ordinary one-way ANOVA and the two-stage linear step-up procedure of Benjamini, Krieger and Yekutieli, with a single pooled variance. *, P≤0.05; **, P≤0.01; ***, P≤0.001 and ****, P≤0.0001. Averaged data are presented as the mean±SEM.

FIG. 16. B cells express high Δ42PD-1 after BCR-stimulation and during HIV-1 infection. Δ42PD-1 breaks BCR-triggered cell cycle progress through the recruitment of SHP1. SHP1 binds and inhibits AKT1 activation, suppressing the AKT1/FOXO1 pathway. Δ42PD-1-specific antibody restores the proliferation of B cells from HIV-1 patients.

### BRIEF DESCRIPTION OF THE SEQUENCES

SEQ ID NO: 1: CH101 VH amino acid sequence

SEQ ID NO: 2: CH101 VK amino acid sequence

SEQ ID NO: 3: hufrCH101 VH amino acid sequence

SEQ ID NO: 4: hufrCH101 VK amino acid sequence

SEQ ID NO: 5: nucleotide sequence encoding CH101 VH

SEQ ID NO: 6: nucleotide sequence encoding CH101 VK

SEQ ID NO: 7: nucleotide sequence encoding hufrCH101 VH

SEQ ID NO: 8: nucleotide sequence encoding hufrCH101 VK

SEQ ID NO: 9: human germline variable heavy chain amino acid sequence

SEQ ID NO: 10: human germline variable kappa chain amino acid sequence

SEQ ID NO: 11: J region of human heavy chain amino acid sequence

SEQ ID NO: 12: J region of human kappa chain amino acid sequence

SEQ ID NO: 13: nucleotide sequence encoding human germline variable heavy chain

SEQ ID NO: 14: nucleotide sequence encoding human germline variable kappa chain

SEQ ID NO: 15: nucleotide sequence encoding J region of human heavy chain

SEQ ID NO: 16: nucleotide sequence encoding J region of human kappa chain

SEQ ID NO: 17: human kappa chain constant fragment nucleic acid sequence (IgG1 and IgG4)

SEQ ID NO: 18: human kappa chain constant fragment amino acid sequence (IgG1 and IgG4)

SEQ ID NO: 19: human heavy chain constant fragment amino acid sequence (IgG1)

SEQ ID NO: 20: human heavy chain constant fragment nucleic acid sequence (IgG1)

SEQ ID NO: 21: human heavy chain constant fragment amino acid sequence (IgG4)

SEQ ID NO: 22: human heavy chain constant fragment nucleic acid sequence (IgG4)

SEQ ID NO: 23: CH101 VH CDR1 amino acid sequence

SEQ ID NO: 24: CH101 VH CDR2 amino acid sequence

SEQ ID NO: 25: CH101 VH CDR3 amino acid sequence

SEQ ID NO: 26: CH101 VL CDR1 amino acid sequence

SEQ ID NO: 27: CH101 VL CDR2 amino acid sequence

SEQ ID NO: 28: CH101 VL CDR3 amino acid sequence

SEQ ID NO: 29: CH34 VH CDR1 amino acid sequence

SEQ ID NO: 30: CH34 VH CDR2 amino acid sequence

SEQ ID NO: 31: CH34 VH CDR3 amino acid sequence

SEQ ID NO: 32: CH34 VL CDR1 amino acid sequence

SEQ ID NO: 33: CH34 VL CDR2 amino acid sequence

SEQ ID NO: 34: CH34 VL CDR3 amino acid sequence

SEQ ID NO: 35: Mouse CH34 VH amino acid sequence

SEQ ID NO: 36: Mouse CH34 VL amino acid sequence

SEQ ID NO: 37: Humanized CH34 VH amino acid sequence

SEQ ID NO: 38: Humanized CH34 VL amino acid sequence

SEQ ID NO: 39: Signal peptide sequence

SEQ ID NO: 40: Mouse CH34 heavy chain amino acid sequence

SEQ ID NO: 41: Mouse CH34 kappa chain amino acid sequence

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts the term “about” is provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a therapeutic composition. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder; immune health; enhancement of the function of enamel, an organ, tissue, or system in the body. The preferred subject in the context of this invention is a human. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of an antibody or composition that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the term “treatment” refers to eradicating, reducing, ameliorating, or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

In some embodiments of the invention, the method comprises administration of multiple doses of the antibody compositions of the subject invention. The method may comprise administration of 1, 2, 3, 4, 5, 6, 7, 8, 9, 10, 15, 20, 25, 30, 35, 40, or more therapeutically effective doses of a composition comprising the antibodies of the subject invention as described herein. In some embodiments, doses are administered over the course of 1 day, 2 days, 3 days, 4 days, 5 days, 6 days, 7 days, 10 days, 14 days, 21 days, 30 days, or more than 30 days. Moreover, treatment of a subject with a therapeutically effective amount of the antibodies of the invention can include a single treatment or can include a series of treatments. It will also be appreciated that the effective dosage of an antibody used for treatment may increase or decrease over the course of a particular treatment. Changes in dosage may result and become apparent from the results of diagnostic assays or imaging techniques for detecting tumor sizes known in the art. In some embodiments of the invention, the method comprises administration of the antibodies at several time per day, including but not limiting to 2 times per day, 3 times per day, and 4 times per day.

The disclosure includes all amino acid sequences described herein, and all polynucleotides encoding the amino acid sequences. The disclosure also includes all amino acid sequences that have at least 80% similarity to any amino acid sequence described herein. The percent amino acid sequence can be determined across the full length of any amino acid sequence described herein, or across any contiguous amino acid sequence that constitutes a functional antibody, such as huCH101, non-limiting examples of which are provided below. As such, the disclosure includes sequences that are the same as any amino acid or nucleotide sequence described herein, and from 80, 81, 82, 83, 84, 85, 86, 87, 88, 89, 90, 91, 92, 93, 94, 95, 96, 97, 98, 99% identical to such sequences.

As used herein, the term “humanized” refers to antibodies from non-human species, such as, for example, mice, that have been modified to increase their similarity to antibody variants produced naturally in humans.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Humanized Δ42PD1-Specific mAbs

The present invention provides antibodies that target the alternatively spliced isoform of PD-1 (herein referred to as “Δ42PD1”). In certain embodiments, the antibody is a monoclonal antibody. In certain embodiments, variable regions, including variable regions of the heavy and/or light chains, of an antibody that targets to Δ42PD1 can be linked to a human antibody constant fragment, including, for example, human IgG4 constant fragment (SEQ ID NO: 21), or preferably, human IgG1 constant fragment (SEQ ID NO: 19). In certain embodiments, the antibody that targets Δ42PD1 is murine CH101 or murine CH34. CH101 and CH34 has been previously described: (see U.S. Pat. No. 10,047,137 and U.S. application Ser. No. 16/030,180, each of which is hereby incorporated in its entirety by reference). The complementarity-determining regions of the antibodies of the subject invention can be derived from the murine CH101 or CH34 hybridoma and the framework regions are derived from a human germline.

The variable regions of CH101 can be linked with the human IgG1 constant fragment, and the constructed chimeric CH101 can be present a humanized rate of about 66.1% (437/661). Optionally, the antibody can be further humanized. Specifically, the variable regions of chimeric CH101 can be altered to increase the humanized proportion to about 97.0% (641/661), resulting in a humanized CH101 antibody (huCH101). The variable regions of CH34 can be linked with the human constant fragment, e.g., human IgG1 or IgG4 constant fragment and/or human kappa constant fragment. In certain embodiments, complementarity-determining region (CDR) grafting can be utilized to alleviate immunogenicity. The approach to fulfill CDR grafting is to determine the CDR regions of non-human antibodies firstly. Secondly, human germlines can be searched for the most homologous ones suitable as a receptor. Finally, the murine CDRs can be transplanted into human framework regions (FRs) correspondingly.

Accordingly, the present disclosure provides a Δ42PD1-specific antibody or an antigen binding fragment thereof, comprising a heavy chain variable region (VH) and a light chain variable region (VL), wherein
(i) the VH comprises HCDRs 1-3 having the amino acid sequences as shown in SEQ ID NOs: 23-25 respectively, and the VL comprises HCDRs 1-3 having the amino acid sequences as shown in SEQ ID NOs: 26-28 respectively; or
(ii) the VH comprises HCDRs 1-3 having the amino acid sequences as shown in SEQ ID NOs: 29-31 respectively, and the VL comprises LCDRs 1-3 having the amino acid sequences as shown in SEQ ID NOs: 32-34 respectively.

The present disclosure can use CDRs defined according to any of these numbering systems, although preferred embodiments use Kabat or Chothia defined CDRs.

When CDR sequences are defined according to Kabat numbering system, the VH of the antibody disclosed herein comprises HCDR1, HCDR2 and HCDR3 having the amino acid sequences as shown in SEQ ID NO: 23 (GYSITSGF), SEQ ID NO: 24 (HYSGS) and SEQ ID NO: 25 (SDF) respectively, and the VL of the antibody disclosed herein comprises LCDR1, LCDR2 and LCDR3 having the amino acid sequences as shown in SEQ ID NO: 26 (TSSQSLLDRDGETFMN), SEQ ID NO: 27 (LVSKLDF) and SEQ ID NO: 28 (WQGTHFPL) respectively; or
the VH of the antibody disclosed herein comprises HCDR1, HCDR2 and HCDR3 having the amino acid sequences as shown in SEQ ID NO: 29 (GFTFSDYG), SEQ ID NO: 30 (ISSLAYTI) and SEQ ID NO: 31 (ARGGAGFAY) respectively, and the VL of the antibody disclosed herein comprises LCDR1, LCDR2 and LCDR3 having the amino acid sequences as shown in SEQ ID NO: 32 (DHINNW), SEQ ID NO: 33 (GAT) and SEQ ID NO: 34 (QQYWSTPWT) respectively.

In some embodiments, the VH comprises an amino acid sequence having at least 80%, at least 85%, at least 90%, at least 95%, at least 98%, at least 99%, or 100% sequence identity to SEQ ID NO: 1 and the VL comprises an amino acid sequence having at least 80%, at least 85%, at least 90%, at least 95%, at least 98%, at least 99%, or 100% sequence identity to SEQ ID NO: 2, or

In some embodiments, the VH comprises an amino acid sequence having at least 80%, at least 85%, at least 90%, at least 95%, at least 98%, at least 99%, or 100% sequence identity to SEQ ID NO: 3 and the VL comprises an amino acid sequence having at least 80%, at least 85%, at least 90%, at least 95%, at least 98%, at least 99%, or 100% sequence identity to SEQ ID NO: 4.

In some embodiments, the VH comprises an amino acid sequence having at least 80%, at least 85%, at least 90%, at least 95%, at least 98%, at least 99%, or 100% sequence identity to SEQ ID NO: 35 and the VL comprises an amino acid sequence having at least 80%, at least 85%, at least 90%, at least 95%, at least 98%, at least 99%, or 100% sequence identity to SEQ ID NO: 36.

In some embodiments, the VH comprises an amino acid sequence having at least 80%, at least 85%, at least 90%, at least 95%, at least 98%, at least 99%, or 100% sequence identity to SEQ ID NO: 37 and the VL comprises an amino acid sequence having at least 80%, at least 85%, at least 90%, at least 95%, at least 98%, at least 99%, or 100% sequence identity to SEQ ID NO: 38.

In a preferred embodiment, the VH comprises an amino acid sequence as shown in SEQ ID NO: 1 and the VL comprises an amino acid sequence as shown in SEQ ID NO: 2. In another preferred embodiment, the VH comprises an amino acid sequence as shown in SEQ ID NO: 3 and the VL comprises an amino acid sequence as shown in SEQ ID NO: 4. In another preferred embodiment, the VH comprises an amino acid sequence as shown in SEQ ID NO: 35 and the VL comprises an amino acid sequence as shown in SEQ ID NO: 36. In another preferred embodiment, the VH comprises an amino acid sequence as shown in SEQ ID NO: 37 and the VL comprises an amino acid sequence as shown in SEQ ID NO: 38.

In some embodiments, the antibody is of an isotype selected from the group consisting of IgG, IgA, IgM, IgE and IgD. In some embodiments, the antibody is of a subtype selected from the group consisting of IgG1, IgG2, IgG3, and IgG4.

In some embodiments, the antibody comprises a heavy chain constant region comprising an amino acid sequence having at least 80%, at least 85%, at least 90%, at least 95%, at least 98%, at least 99%, or 100% sequence identity to SEQ ID NO: 19 or 21. In some embodiments, the antibody comprises a light chain constant region comprising an amino acid sequence having at least 80%, at least 85%, at least 90%, at least 95%, at least 98%, at least 99%, or 100% sequence identity to SEQ ID NO: 18.

In some embodiments, the antigen binding fragment is selected from the group consisting of Fab, Fab′, F(ab′)₂, Fd, Fd′, Fv, scFv, ds-scFv and dAb.

In some embodiments, the antibody is a murine, human, or humanized antibody. In a preferred embodiment, the antibody is humanized antibody.

In some embodiments, the antibody is a monoclonal antibody.

In some embodiments, the antibody comprises a heavy chain comprising an amino acid sequence having at least 80%, at least 85%, at least 90%, at least 95%, at least 98%, at least 99%, or 100% sequence identity to SEQ ID NO: 40 and a light chain comprising an amino acid sequence having at least 80%, at least 85%, at least 90%, at least 95%, at least 98%, at least 99%, or 100% sequence identity to SEQ ID NO: 41.

In some embodiments, the antibody is a bi-specific or a multi-specific antibody. In some embodiments, the antibody is a bispecific antibody which further comprises a second antigen binding region binding to a second antigen. In some embodiments, the second antigen is a tumor associated antigen or an immune cell antigen, e.g., a T-cell antigen.

In certain embodiments, the humanized Δ42PD1-specific antibody comprises a variable heavy chain with SEQ ID NO: 3 or sequence with at least 70%, 80%, 90%, 95%, 97%, 98%, or 99% sequence identity to SEQ ID NO: 3 and a variable light chain of SEQ ID NO: 4 or sequence with at least 70%, 80%, 90%, 95%, 97%, 98%, or 99% sequence identity to SEQ ID NO: 4. In certain embodiments, the light chain is a kappa light chain. In certain embodiments, the humanized Δ42PD1-specific mAb comprises human IgG1 constant heavy chain fragment, according to SEQ ID NO: 19 or a sequence having at least 70%, 80%, 90%, 95%, 97%, 98%, or 99% identity to SEQ ID NO: 19, and human IgG1 light chain fragment, according to SEQ ID NO: 18 or a sequence having at least 70%, 80%, 90%, 95%, 97%, 98%, or 99% identity to SEQ ID NO: 18.

In certain embodiments, the humanized Δ42PD1-specific antibody comprises a variable heavy chain encoded by SEQ ID NO: 7 or sequence with at least 70%, 80%, 90%, 95%, 97%, 98%, or 99% sequence identity to SEQ ID NO: 7 and a variable light chain encoded by SEQ ID NO: 8 or sequence with at least 70%, 80%, 90%, 95%, 97%, 98%, or 99% sequence identity to SEQ ID NO: 8. In certain embodiments, the light chain is a kappa light chain. In certain embodiments, the humanized Δ42PD1-specific mAb comprises human IgG1 constant heavy chain fragment, encoded by SEQ ID NO: 20 or a sequence having at least 70%, 80%, 90%, 95%, 97%, 98%, or 99% identity to SEQ ID NO: 20, and human IgG1 light chain fragment, encoded by SEQ ID NO: 17 or a sequence having at least 70%, 80%, 90%, 95%, 97%, 98%, or 99% identity to SEQ ID NO: 17.

Variable regions of either heavy or light chain comprise three CDRs and four FRs. The CDRs are the main contact regions with antigens, thus retaining the main function of an antibody. In certain embodiments, the percentage of sequence variation in the FR regions, according to, for example, SEQ ID NO: 9 and/or SEQ ID NO:10 can amount to about 10% and retain ability to bind to Δ42PD1. In the case of humanized CH101, we kept all the CDRs of parental CH101, and tested the function alteration after humanization. The result showed that hufrCH101 retained 76.79% function of parental murine CH101 (Table 1). The average sensitivity of hufrCH101-IgG1 can be about 45.46% against 293T-Δ42PD1 cells, and the parental murine CH101 average sensitivity can be about 59.2% against 293T-Δ42PD1 cells. This association can be interpreted that hufrCH101-IgG1 retains about 76.79% function of parental murine CH101.

In preferred embodiments, the compositions and methods according to the subject invention utilize a humanized Δ42PD1-specific mAb. A humanized Δ42PD1-specific mAb may be added to compositions at concentrations of 0.01 to 90% by weight (wt %), preferably 0.1 to 50 wt %, and more preferably 0.1 to 20 wt %. In another embodiment, a purified a humanized Δ42PD1-specific mAb may be in combination with an acceptable carrier, in that a humanized Δ42PD1-specific mAb may be presented at concentrations of 0.001 to 50% (v/v), preferably, 0.01 to 20% (v/v), more preferably, 0.02 to 10% (v/v). In certain embodiments, the humanized Δ42PD1-specific mAb can be administered to a subject at a dosage of about 1 mg/kg to about 100 mg/kg.

In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

In one embodiment, the compositions of the subject invention can be made into aerosol formulations so that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the compositions of the subject invention can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

### Administration of Humanized Δ42PD1-Specific mAb

In certain embodiments, a humanized Δ42PD1-specific mAb can be administered to a subject. Any means of administration that can permit an inhibitor to contact cells in a subject, including, for example, orally, intravenously, intraperitoneally, intramuscularly, intrathecally, or subcutaneously are envisioned in the subject methods. In preferred embodiments, a humanized Δ42PD1-specific mAb can be administered intravenously.

In certain embodiments, a humanized Δ42PD1-specific mAb can contact healthy cells of subject and/or tumor cells or cancerous cells. In certain embodiments, a humanized Δ42PD1-specific mAb can contact cells in the liver, including cancerous liver cells. In certain embodiments, a humanized Δ42PD1-specific mAb can inhibit hepatocellular carcinoma and other solid tumors. The humanized Δ42PD1-specific mAb can permeate the cells of a subject, including, for example, cancerous cells. In certain embodiments, the humanized Δ42PD1-specific mAb can inhibit the growth of tumor cells. The humanized Δ42PD1-specific mAb can inhibit the growth of tumor cells by modulating the Δ42PD1 expression and/or the function of Δ42PD1-TLR4 axis.

### Materials and Methods

### Plasmid Construction

For the expression plasmid construction, we used the NEBuilder® HiFi DNA Assembly Master Mix (New England BioLabs, Ipswich, MA) to assemble the variable heavy chain of the antibody with the backbone of AbVec2.0-IGHG1 plasmid (Addgene, Watertown, MA). And the variable kappa chain of the antibody was assembled with the backbone of AbVec1.1-IGKC plasmid (Addgene).

### Antibody Detection

Mice plasma was diluted with PBS at 1:1 ratio for measurement of inflammatory cytokine profiles using LEGENDplex™ Human Inflammation panel 1 (13-plex) (Biolegend, San Diego, CA) according to manufacturer's instruction. BioLegend's LEGENDplex™ assays are bead-based immunoassays using the same basic principle as sandwich immunoassays. The concentration of a particular analyte is determined using a standard curve generated in the same assay (see worldwide website: biolegend.com/en-us/products/legendplex-human-inflammation-panel-1-13-plex-with-v-bottom-plate-16929?GroupID=GROUP25, which is hereby incorporated by reference along with the current manual).

### Statistical Analysis

GraphPad Prism 6.0 (GraphPad Software, San Diego, CA) was applied for analyzing statistics in this study. Ordinary one-way ANOVA as well as multiple comparisons were performed to compare the group means when more than two groups were involved. The difference of group means within two groups only were assessed by unpaired t test. A p-value<0.05 was significant. Specific details on statistical tests were denoted in the figure legends.

### Patients, Treatment, and Sample Collection

Healthy human blood buffy coats were obtained from the Red Cross of Hong Kong from donors at median age of 31 (interquartile range, 22-67; 62.16% male versus 37.84% female; HBV/HCV negative). Eligible patients were 18 years of age or older, with the diagnosis confirmed by histologic or cytologic analysis with clinical features according to the American Association for the Study of Liver Diseases. All patients had not previously received systemic therapy for liver cancer before enrolment. 41 patients were administrated in the Department of Surgery, the Queen Mary Hospital, Hong Kong, who were subjected to curative resection as the standard therapy from July 2016 to November 2019. Sample were collected at two time points: peripheral whole blood were collected before surgical resection, paired tumor and adjacent non-tumor samples were collected after resection.

### Flow Cytometric and Immunofluorescence Analysis

Flow cytometric analyses and immunofluorescence analyses were performed and analyzed as described previously. Cells were incubated for 10 min with Fc Block (Biolegend) in staining buffer (PBS containing 2% FBS) followed by staining with the indicated antibodies (Supplementary Table 2) for 30 min at 4° C. For intracellular staining, cells were fixed and permeabilized with BD Cytofix/Cytoperm (BD Biosciences) prior to staining with mAbs in Perm/Wash buffer (BD Biosciences) for 30 min at 4° C., and washed with 1 ml of staining buffer. All samples were acquired using a FACSAria III flow cytometer (BD Biosciences), and results were analyzed with FlowJo software (v10.6) (BD Biosciences).

The following antibodies were purchased from BioLegend and used for flow cytometry: CD3 (clone HIT3a), CD4 (clone A161A1), CD8a (clone HIT8a), CD45 (clone 2D1), CD19 (clone HIB19), CD16 (clone 3G8), CD14 (clone HCD14), CD11c (clone S-HCL-3), HLA-DR (clone LN3), CD45RA (clone HI100), CCR7 (clone G043H7), CD27 (clone M-T271), IFN-γ (clone B27), TNF-α (MAb11), IL-2 (clone MQ1-17H12), PD-1 (clone EH12.2H7, NAT105 and A17188B), Tim-3 (clone F38-2E2) and LAG3 (clone 11C3C65). The following antibodies were purchased from BD Biosciences and used for flow cytometry: CD56 (NCAM-1), VISTA (clone MIH65) and CD244 (clone 2-69). Δ42PD-1 antibody (clone CH101) was labelled with Alexa Fluor 568/647 using an Antibody Labeling Kit (Invitrogen) as previously described for flow cytometry. Dead cells were stained with Zombie Aqua Fixable Viability Kit (BioLegend). Cell surface and intracellular immunostaining were performed as previously described. Annexin V staining (BD Bioscience) was performed according to the manufacturer's instructions. Flow cytometric data analysis was performed using the FlowJo software (Tree Star, v10).

Immunofluorescent and immunohistochemical staining was performed as reported previously, with anti-human CD3 (clone CD3-12, AbD Serotec; #A0452, DAKO), anti-human TLR4 (#ab150583, Abcam), anti-Δ42PD-1 (clone CH101), anti-IL6 (clone M-19, Santa Cruz) and anti-PD-1 (clone NAT105, Abcam) antibodies, followed by secondary antibodies labelled with either Alexa Fluor 488, Alexa Fluor 568 or Alexa Fluor 647 (Invitrogen). Counterstaining was done by Hoechst 33258, and stained sections were mounted using fluorescence mounting medium (DAKO). The images were then acquired by a confocal microscope (LSM710/780/800, Carl Zeiss, ×20 and ×40 objective lenses) with the ZEN software. The integrated intensity of immunostaining signals and cell counts were quantified by the software ImageJ.

### Humanized Mouse Model

Immunodeficient NOD.Cg-Prkdc^{scid} Il2rg^{tm1Wjl}/SzJ (NSG, Jackson Laboratories) were bred and housed in the minimal disease area at the HKU Laboratory Animal Unit (LAU). All animal experiments were approved by the Committee on the Use of Live Animals in Teaching and Research at the Laboratory Animal Unit of the University of Hong Kong (license #3883-16). Mice with both genders were chosen randomly without blinding. Six to eight-week-old mice were reconstituted with 1.0-1.5×10⁷ healthy human PBMCs by intraperitoneal (i.p) injection in 0.5 ml PBS. At day 10, the animal blood was sampled and examined for the level of human lymphocytes by flow cytometry. Red blood cells in blood samples were lysed with red blood cell lysis buffer (BD PharmLyse) before staining. Mice with successful engraftment (NSG-huPBL) were used for establishing the HCC models. For the subcutaneous (s.c.) HCC model, 2×10⁶Huh7 or PLC5 cells were injected s.c. into right hind flank of each NSG-huPBL mouse. Tumor volumes were measured by caliper (Tumor volume=½(length×width²)). For orthotopic HCC model, s.c. MHCC-97L tumors were harvested from NSG mice when they reached 1 cm in diameter and were cut into pieces under aseptic conditions. Intact pieces with 1 mm³ in size were then implanted into the left liver lobe of each NSG-huPBL mouse. Luciferase-expressing tumors were measured with the IVIS spectrum (PerkinElmer) and presented as photons/s/cm²/sr within regions of interest (ROI) using Living Image software (version 4.0, PerkinElmer) as previously described². Specimens were fixed in Zinc Formalin Fixative (sigma) and then embedded in paraffin blocks.

### Ex Vivo Cell Preparation

Murine PBMCs and splenocytes were isolated as previously described. Fresh PBMCs were isolated by gradient centrifugation (Ficoll-Paque, GE Healthcare) from either healthy or HCC patients' peripheral blood. Tumors or adjacent non-tumor tissues were cut into pieces and digested with Tumor Dissociation Kit (Miltenyi Biotec) according to the manufacturer's instructions under a gentleMACS Octo Dissociator (Miltenyi Biotec). Cells were passed through a 70 μm strainer and then subjected to 40%/80% Percoll gradient (Sigma). Leukocytes at the interphase were recovered after centrifugation at ×800 g 20 min. CD3⁺ T cells were isolated using the Pan T Cell Isolation Kit (Miltenyi Biotech). Δ42PD-1⁺ and Δ42PD-1⁻ T cells were isolated by a cell sorter (BD AriaIII) after specific antibody staining.

### In Vitro T Cell Culture, Proliferation Assay and Cytotoxicity Assay

Cells were incubated at 37° C., 5% CO₂ in RPMI 1640 (Gibco) supplemented with 10% FBS (Gibco) and antibiotics in 96-well U-bottom plates. For the proliferation assay, purified T cells were pre-stained with Carboxyfluorescein 6 succinimidyl ester (CFSE, Thermo Scientific) prior to the culture, and were incubated with T Cell Activation/Expansion Kit (Miltenyi Biotec) at a beads-to-T cell ratio of 1:4 for 4-5 days before analysis on flow cytometry (BD AriaIII). Culture supernatant was harvested for analysis of cytokine production. Cytotoxic effect of purified T cells against Huh7 cells was determined using NonRadioactive Cytotoxicity Assay (Promega) according to the manufacturer's instructions. All the cytokine concentrations in plasma, culture supernatant or tumor lysates were measured by LEGENDplex Human Panel (13-plex, BioLegend). Tumors were cut into pieces and homogenized in T-PER Tissue Protein Extraction Reagent (Thermo Scientific) supplemented with Protease Inhibitor Cocktail (Roche) before analysis.

### HIV-1 Infected Subjects

We studied a total of 86 HIV-1 infected patients recruited at the Shenzhen Third People's Hospital, Shenzhen, Guangdong province, China, including 12 patients on ART and 74 untreated patients. The study was conducted with the approval of Institutional Review Board of University of Hong Kong/Hospital Authority Hong Kong West Cluster, Hong Kong East Cluster Research Ethics Committee, and Kowloon West Cluster Research Ethics Committee (UW 19-834). Blood samples were collected after written informed consent was obtained from each participant. All patients were seronegative for hepatitis B and C. None showed signs of active tuberculosis.

### Peripheral Blood Mononuclear Cell (PBMC) Isolation

PBMCs were isolated from fresh blood samples collected from patients and healthy donors using Ficoll-Paque density gradient centrifugation in our BSL-3 laboratory. The purified PBMCs were used for immune cell phenotyping and B cell isolation. The remaining cells were cryopreserved in freezing medium (90% FBS+10% DMSO) at a density of 5×10⁶ cells/mL at −150° C.).

### Antibody Purification

Δ 42PD1-specific monoclonal antibody CH101 and CH34 are purified as previously described (Cheng et al., 2015; Cheung et al., 2017). In Brief, CH34 or CH101 producing hybridoma cells are intraperitoneally injected into incomplete Freund's adjuvant primed 6-8 weeks old BALB/c mice. After one to two weeks, ascites fluid is withdrawn from the peritoneal cavity, and incubated at 37 degrees for 1 hour, followed by 4 degrees overnight. The ascites fluid is then centrifuged at 4000×g for 30 min at 4 degrees, and diluted with 1-2 parts of PBS for antibodies purification using Pierce™ Protein G Agarose. Anti-Δ42PD1 antibodies CH34 were labelled with Alexa Fluor 647 using an Antibody Labeling Kit (Invitrogen).

### Immunocytochemistry Assay

Cells were harvested and then concentrated to 3×10⁶ cells/ml. 10 d cell suspensions were dropped on clean glass slides followed by drying in a hood for 1 h. Samples were fixed in either cold methanol or 10% neutral buffered formalin for 10 min at RT and permeabilized with 0.5% Triton X-100 for 10 min. Samples were then stained with primary antibody for 1 h at RT, followed by staining with fluorescein conjugated secondary antibody for 1 h at RT after washing with PBS. Cells were incubated with 1:10,000 diluted Hoechst33258 (Cat. No. H3569, Life Technologies) for 10 min at RT. After washing, microscopic images were obtained using a Carl Zeiss LSM 780 confocal microscope (Oberkochen, Germany).

### B Cell Isolation

Total B cells were isolated from human PBMCs using a pan B cell isolation kit (Miltenyi Biotec) followed by IgD-positive selection using anti-IgD microbeads (Miltenyi Biotec) according to the manufacturer's instructions.

### B Cell Culture

B cells were cultured with RPMI 1640 medium containing 10% FBS and 1% streptomycin/penicillin (all from Gibco) for 2-5 days in the presence of α-IgM/G F(ab′)2 (10 μg/ml, Thermofisher) alone, or α-IgM/G F(ab′)2 plus CpG (1 μM, Invitrogen) or α-IgM/G F(ab′)2 (10 μg/ml) plus IL-2 (20 U/ml, R&D Systems), IL-10 (100 ng/ml, R&D Systems) and CD40L (500 ng/ml, PeproTech). To determine the B cell proliferation, Carboxyfluorescein-6-succinimidyl ester (CFSE, 5 μM in PBS, Biolegend)-labeled B cells were cultured in 96-well U-bottom plates for 4-5 days. Proliferating B cells were determined by calculating the percentage of CFSE-low cells. For the immunoprecipitation/Co-IP and Western Blot assay, B cells were cultured for 2 days. For the experiment with CH34 treatment, 10 μg/mL of CH34 or isotype control msIgG2b was added to B cells at day 0.

### Immunoprecipitation/Co-IP and Western Blot Assay

For cell lysis, we used 1% SDS lysis buffer (50 mM Tris, pH 8.0, 1 mM EDTA, 100 mM NaCl, 5 mM DTT, and 1% SDS) containing protease and phosphatase inhibitors (Thermo Fisher Scientific). The prepared protein lysate samples were incubated with related immunoprecipitation antibody and shaking overnight at 4° C., followed by protein G agarose beads incubation, shaking for another 3-4 hours. Precipitates were washed 4-5 times with lysis buffer. Finally, add SDS loading buffer, boil for 5 minutes and subjected for SDS-PAGE electrophoresis. Immuno-precipitate or protein lysates were separated by SDS-PAGE using 8% or 12% polyacrylamide gel (80V, ˜30 min, 120V, ˜1.2 h) and then transferred to a polyvinylidene difluoride (PVDF) membrane (0.3A, 1.2 h). The transferred membrane was then blocked in 5% skim milk (dissolved in TBST) at room temperature for 1 hour. After blocking, the membrane was incubated with primary antibody solution (˜ 1/1000 diluted in 5% BSA TBST buffer) overnight, at 4° C. Then the membrane was washed with TBST for 3× 10 min. After washing, the membrane was incubated with fluorescence conjugated secondary antibody at room temperature and shake for 1.5 hours. Then washing the membrane again with TBST, 3× 10 min. later dry the membrane and do the scanning by Li-COR Odyssey.

### RNA Isolation, PCR and RT-PCR

Total cellular RNA was extracted by RNAiso plus or All Prep DNA/RNA mini kit according to the instructions of the manufacturer. RNA extraction was directedly used to synthesize the cDNA by PrimeScript RT reagent Kit. cDNA was used to amplify the certain gene by PCR or quantify the related gene expression level by qPCR. qPCR reaction was prepared with SYBR Premix Ex Taq II (Takara) and performed on AB VIIA7 RUO software (Applied Biosystems). Gapdh was used as a housekeeping gene.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Amplification and Construction of huCH101

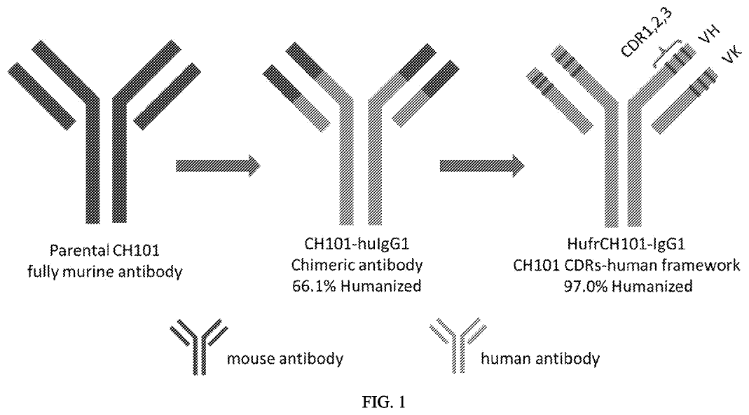

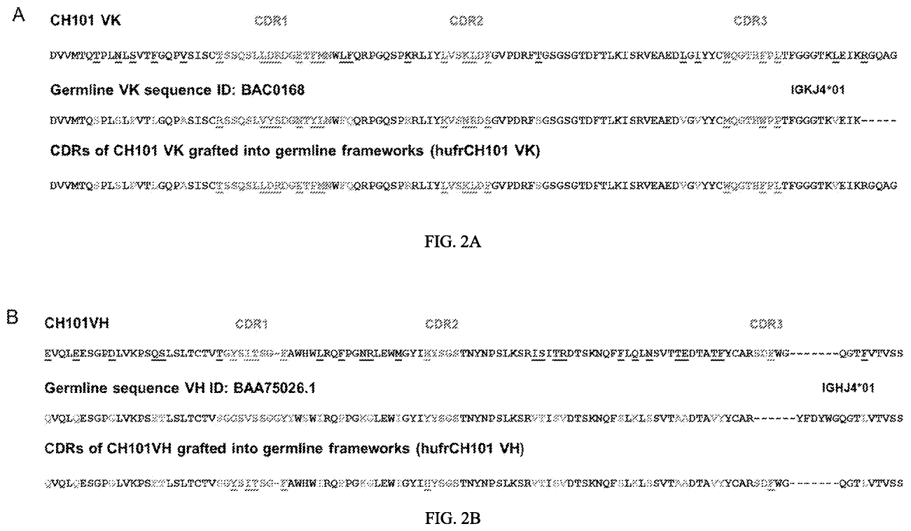

In order to engineer the humanized CH101 antibodies and its variants, the variable heavy and light chains of murine CH101 were amplified from the selected hybridoma clone of CH101 using specific sets of primers as described previously. The cloned sequences were searched in the database of IMGT and V-BASE and aligned with the most compatible human germlines that were selected for CDR grafting, as indicated in FIG. 1 and FIGS. 2A-2B. There are a total number of 112 amino acids in the variable heavy or kappa chain, 330 amino acids in the constant fragments of human IgG1, and 107 amino acids in the constant region of kappa chain. When the variable regions of CH101 were linked with the human IgG1 constant fragment, the constructed chimeric CH101 would present a humanized rate at 66.1% (437/661). We then further humanized the variable regions of chimeric CH101 and increased the humanized proportion to 97.0% (641/661) in huCH101 (or called human framework hufrCH101) (FIG. 1).

### Example 2—Engineering of Humanized Ch101 and its Variants

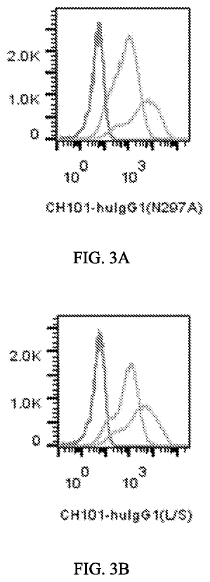

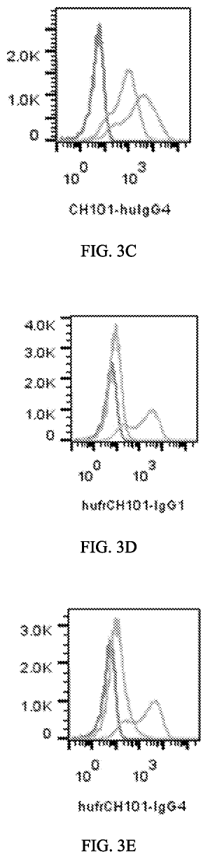

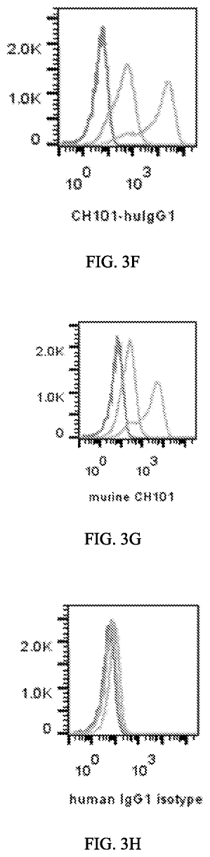

To exploit the therapeutic activities of CH101 variants, we made various modifications in the constant regions of the chimeric version or humanized version of CH101, thus resulting a serial of CH101 humanized antibodies, including CH101-huIgG1(N297A), CH101-huIgG1(L/S), CH101-huIgG4, huCH101-IgG1, huCH101-IgG4, CH101-huIgG1 (chimeric CH101). The original CH101 (parental, fully murine) was included as the positive control for these humanized versions. Due to the CH101-specific binding to the conformational structure of Δ42PD1 when expressed on cell surface, 293T-Δ42PD1 and 293T-PD1 cell lines were established in house for assessment of various versions of humanized CH101 antibodies by flow cytometry. All humanized antibodies could recognize Δ42PD1 expressed on the cell surface but with different specificity (FIGS. 3A-3I). The detailed statistical analysis elucidated the activity of versions of humanized antibodies in interacting with Δ42PD1 at different specificity and sensitivity (Table 1). As the data indicated, the parental murine CH101 could detect an average of 59.20% 293T-Δ42PD1 cells and the sensitivity would increase to 70.08% for the chimeric IgG1 version but decrease to 45.46% for the hufrCH101-IgG1. However, the cross-reaction rates of CH101-huIgG1 with 293T-PD1 cells were at an average of 7.68%, and 3.15% for parental murine CH101. In contrast, there was barely any cross reaction between huCH101-IgG1 with 293T-PD1 cells. Due to the limited expression of chimeric versions of CH101 with Fc engineering, including CH101-huIgG1(N297A), CH101-huIgG1(L/S) and CH101-huIgG4, the amount of these antibody was insufficient for fluorescence labelling. When the FACS staining was conducted using a second anti-human Fc antibody, high frequencies of non-specific staining were observed, leading to non-specific cross-reaction with 293T-PD1 cells. These antibodies, therefore, required further optimization for better production.

TABLE 1
Binding of Δ42PD1 by humanized
CH101 antibodies by flow cytometry

| Antibody (10 ng/ml) | 293T-Δ42PD1 (%) | 293T-PD1 (%) |
|---|---|---|
| CH101-huIgG1(N297A) | 68.29 | 36.09 |
| CH101-huIgG1(L/S) | 65.69 | 38.49 |
| CH101-huIgG4 | 62.79 | 32.89 |
| hufrCH101-IgG4 | 54.59 | 1.58 |
| hufrCH101-IgG1 | (45.46 ± 18.70) | (0.04 ± 0.07) |
| CH101-huIgG1 | (70.08 ± 17.02) | (7.68 ± 6.08) |
| murine CH101 | (59.20 ± 19.84) | (3.15 ± 6.18) |
| human IgG1 isotype | — | — |
| anti-PD1-APC | — | (81.68 ± 14.25) |

### Example 3—Half-Life of Humanized Versions of Ch101 in NSG-PBL Mice

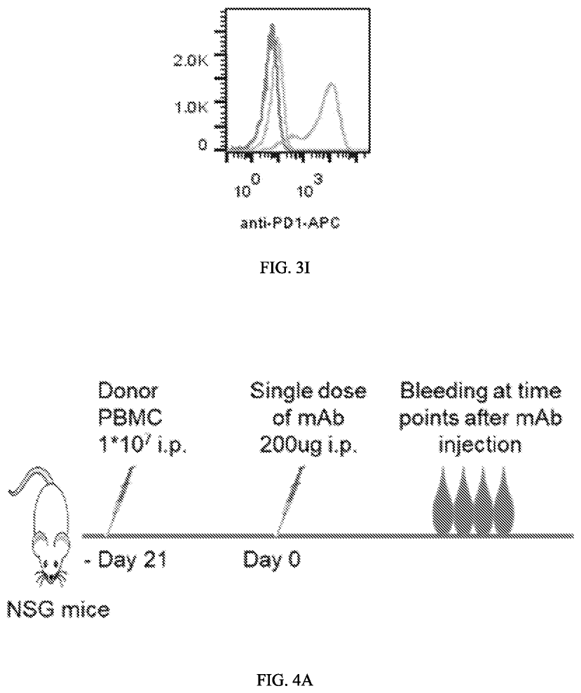

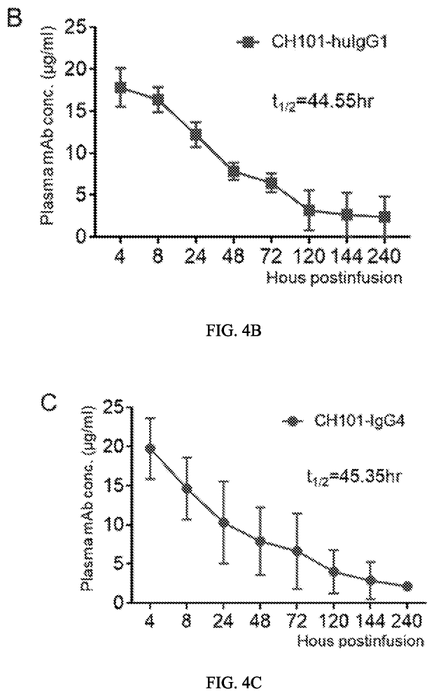

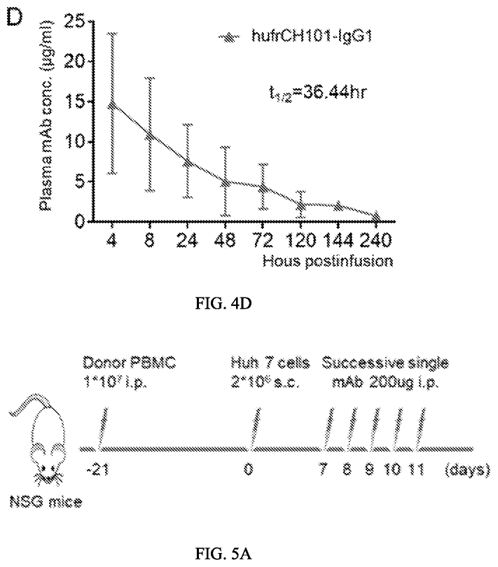

We next assessed three humanized CH101 antibodies, CH101-huIgG1, CH101-IgG4 and hufrCH101-IgG1 in the NSG-PBL mice. We analyzed mice plasma samples obtained at different time intervals after one injection of each individual antibody at the dose of 200 μg per mouse. The peripheral amounts of each antibody were measured subsequently by ELISA for calculating a detailed value of each antibody's half-life. The plasma levels of all three antibodies under investigation declined over time within 72 hours after the injection and subsequently plateaued at a low level after 5 days. The half-life of CH101-IgG4 was 45.35 hr that was slightly longer than others as compared with CH101-huIgG1 (44.55 hr) and hufrCH101-IgG1 (36.44 hr), as indicated in FIGS. 4A-4D.

### Example 4-Chimeric Ch101 as a Therapeutic Intervention Tested in HCC Mouse Model

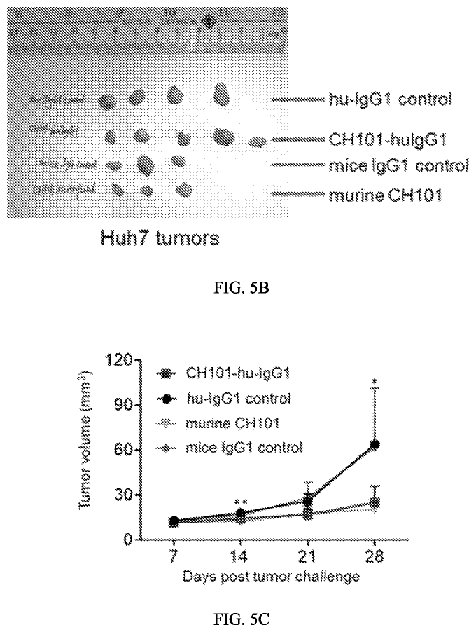

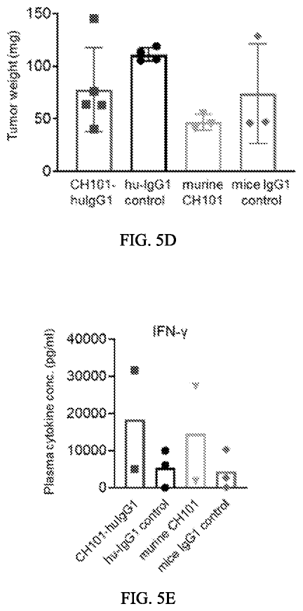

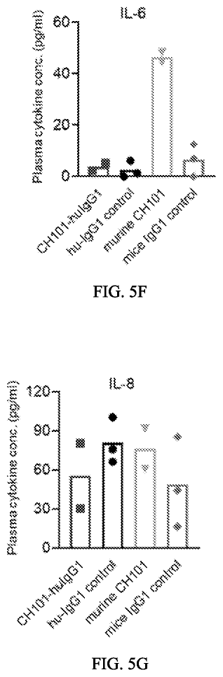

Since the humanized NSG-huCD34 mouse model has not been well established for HIV-1 antibody testing, we firstly assessed the CH101-IgG1, the chimeric version of CH101 (66.1% humanization) for its potential as an immunotherapy in an HCC mouse model. The NSG-PBL mice were established as we previously described. They were then subcutaneously inoculated with Huh7 cells (FIG. 5A). The Huh7 cells grew in NSG-PBL mice for a week before the antibody intervention. After the antibody treatment, we monitor the Huh7 tumor grew over time on a weekly basis until the endpoint (FIG. 5B). On Day 14 and Day 28 (the endpoint) post tumor challenge, there was a significant difference between the antibody treatment and control groups, mainly in the CH101-huIgG1 and the control group (FIG. 5C and Table 2). Specifically, CH101-huIgG1 group displayed an average of 3.83 mm³ smaller tumor than the hu-IgG1 control group on Day 14. At the same time, parental murine CH101 group had a comparable reduction of 4.12 mm³ as compared to the mouse IgG1 control group. At the endpoint, a significant reduction in tumor volume was consistently observed in the CH101-huIgG1 group at an average of 38.94 mm³ (Table 2). However, there was no significant difference either in the tumor weight (FIG. 5D) due to escape in ⅕ mice or in the inflammatory cytokines released into plasma (FIGS. 5E-5G).

TABLE 2
Tumor growth post CH101-huIgG1 treatment in NSG-PBL mice (mm³)

| Antibody | Day 7 | Day 14 | Day 21 | Day 28 |
|---|---|---|---|---|
| CH101-huIgG1 | 11.68 ± 1.22 | 14.30 ± 2.48 | 16.82 ± 6.34 | 24.96 ± 11.12 |
| Human IgG1 control | 12.72 ± 0.96 | 18.13 ± 2.72 | 25.79 ± 5.07 | 63.90 ± 2.68 |
| Murine CH101 | 11.55 ± 0.49 | 12.40 ± 0.61 | 17.83 ± 4.88 | 20.64 ± 4.70 |
| Mouse IgG1 control | 12.39 ± 0.67 | 16.52 ± 0.66 | 28.02 ± 10.55 | 62.13 ± 39.26 |
| CH101-huIgG1 vs. | p = 0.2244 | p = 0.0168 | p = 0.1892 | p = 0.0256 |
| human IgG1 control | | | | |
| Murine CH101 vs. | p = 0.3836 | p = 0.0100 | p = 0.1908 | p = 0.0517 |
| mouse IgG1 control | | | | |
| CH101-huIgG1 vs. | p = 0.9934 | p = 0.3468 | p = 0.9946 | p = 0.9847 |
| murine CH101 | | | | |

Average volume of each group was shown as Mean ± SD. Multiple comparison of the tumor volumes between groups was done and the p values were included.

### Example 5—the hufrCH101-IgG1 as a Therapeutic Intervention Tested in HCC Mouse Model

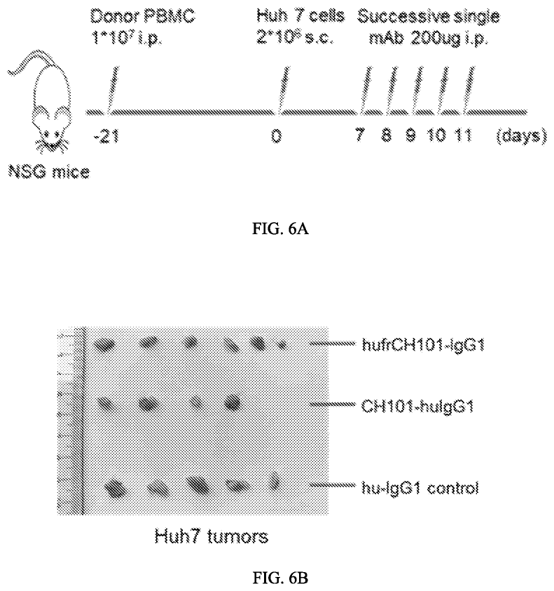

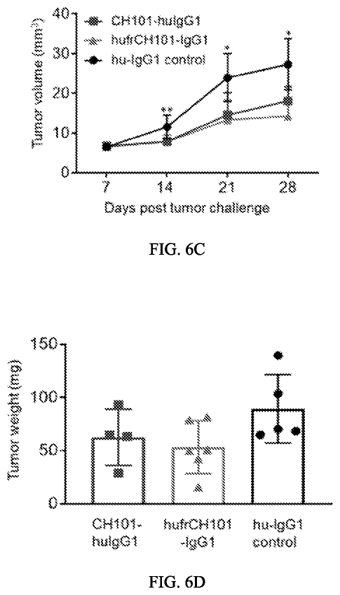

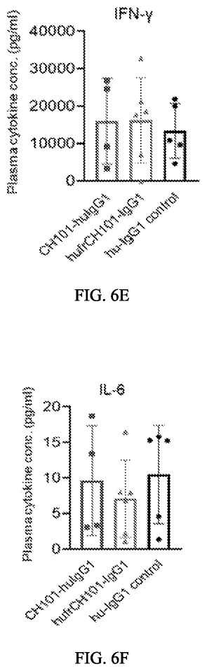

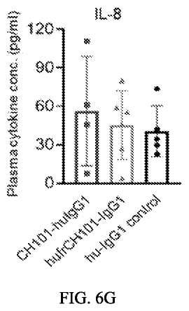

We then evaluated the hufrCH101-IgG1, the 97.0% humanized version of CH101, for suppressing HCC in NSG-PBL mice. Similarly, the NSG-PBL mice were set up and subcutaneously challenged with Huh7 tumor cells (FIG. 6A). The animals received daily antibody treatment for 5 days one week after the tumor inoculation. The tumor growth in mice was measured and the volume of tumor was recorded every week until the humane endpoint (FIGS. 6B-6C). Encouragingly, the tumor size significantly reduced mainly in the hufrCH101-IgG1 group as compared with the control group (FIG. 6C and Table 3). The HufrCH101-IgG1 group presented an average of 7.82 mm³ in tumor size similar to 7.90 mm³ in the CH101-huIgG1 group as compared with 11.55 mm³ in the hu-IgG1 control group on day 14. The tumor size progressed slower in two humanized CH101 antibody groups, showing 13.33 mm³ and 14.56 mm³ in hufrCH101-IgG1 and CH101-huIgG1 on day 21, respectively, which were contrasted with 23.96 mm³ in the control group. Subsequently, hufrCH101-IgG1 still showed a relatively better control of tumor size at the end point, with an average volume of 14.26 mm³ as compared with 27.27 mm³ in the control group (Table 3). Significant decline was found neither with the tumor weight (FIG. 6D) nor with the plasma inflammatory cytokines level (FIGS. 6E-6G), which required further analysis.

### Example 6—Role of Δ42PD-1 in HCC Tumorigenic and Resistance to ICB

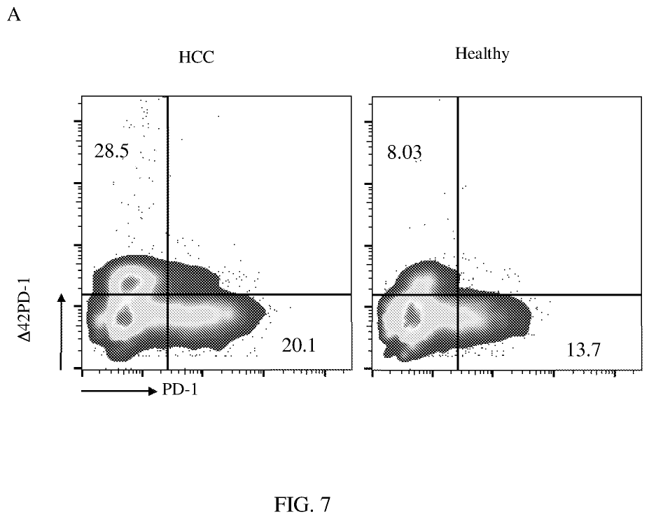

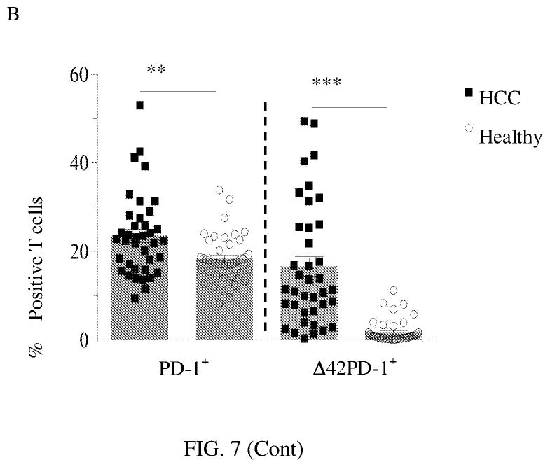

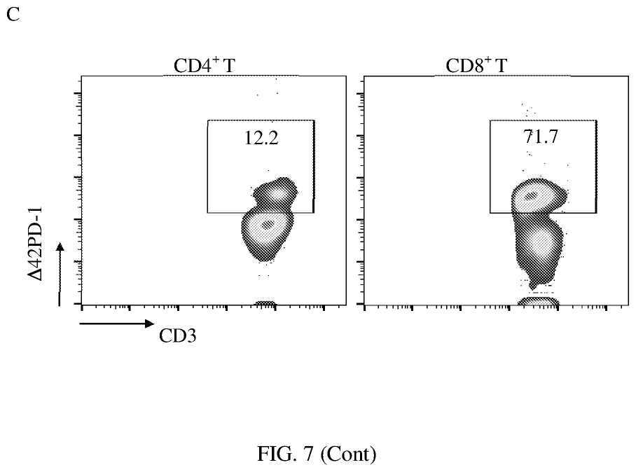

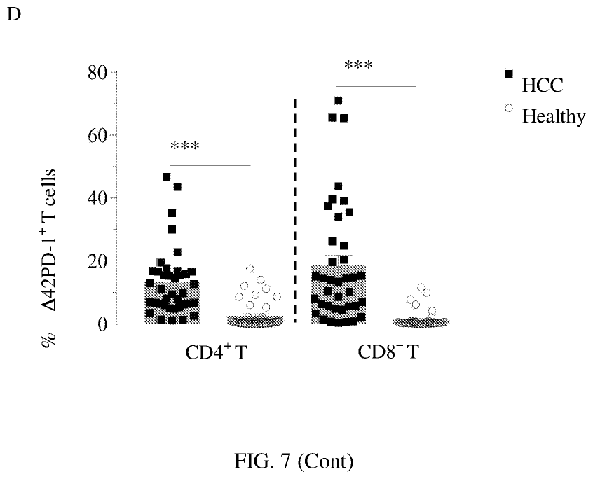

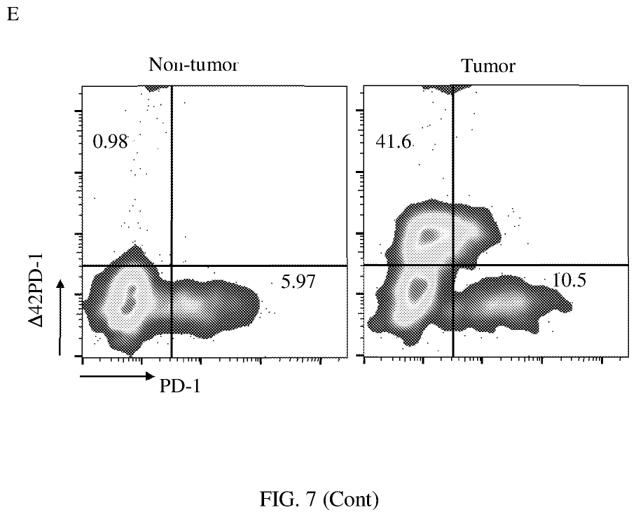

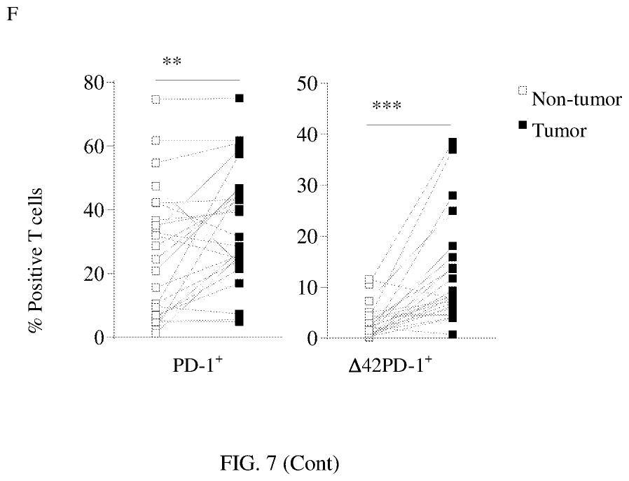

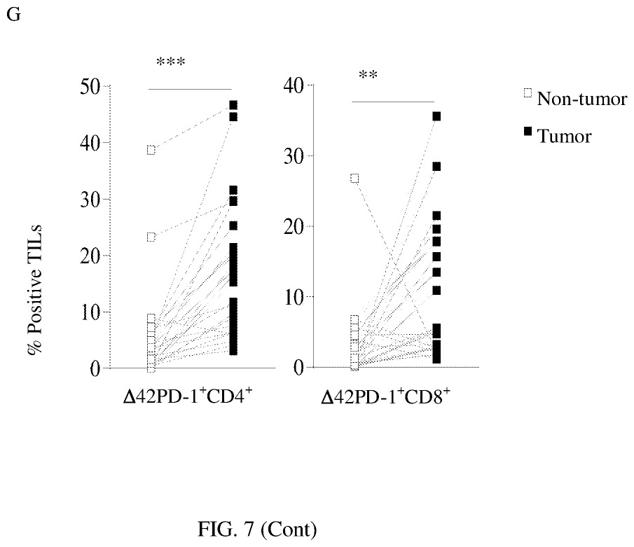

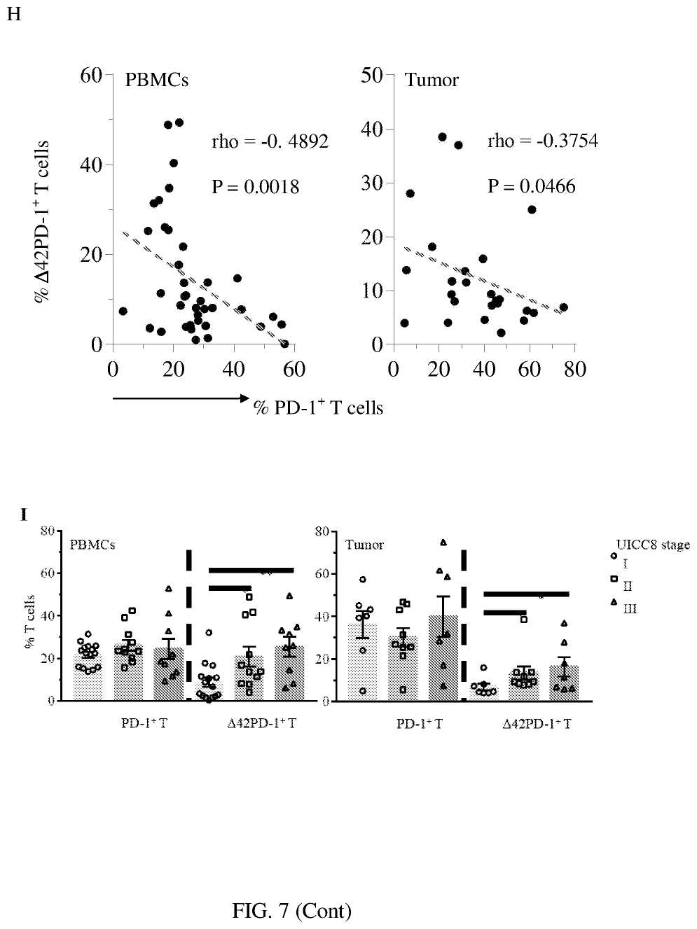

Immune checkpoint blockade (ICB) has improved cancer treatment, yet why most hepatocellular carcinoma (HCC) patients are resistant to PD-1 ICB remains elusive. Here we elucidated the role of a programmed cell death protein 1 (PD-1) isoform, Δ42PD-1, in HCC tumorigenic and resistance to Nivolumab ICB. Δ42PD-1 expression on distinct T cell subsets in HCC patients was detected by the method as described in “MATERIALS AND METHODS” above. We measured Δ42PD-1⁺ and PD-1⁺ T cells in PBMCs of 38 HCC patients and 43 healthy people (FIG. 7A-7B), Δ42PD-1⁺CD4⁺ and CD8⁺ T cells in PBMCs of these HCC patients (FIG. 7C-7D), Δ42PD-1⁺ or PD-1⁺ T cells in paired tumor and adjacent non-tumor tissues derived from HCC patients (FIGS. 7E-7F), and frequencies of Δ42PD-1⁺CD4⁺ or Δ42PD-1⁺CD8⁺ T cells in paired tumor and adjacent non-tumor tissue from the same patients (FIG. 7G). We evaluated correlations between frequencies of Δ42PD-1⁺ and PD-1⁺ T cells in PBMCs (n=38) and tumors (n=25) respectively in the same cohort of HCC patients (FIG. 7H). Frequencies of PD-1⁺ and Δ42PD-1⁺ T cells in PBMCs and tumors of HCC patients stratified into their UICC8 staging group were also measured and shown in FIG. 7I.

We found distinct T cell subsets in untreated HCC patients, which did not express PD-1 but expressed the isoform Δ42PD-1, accounting for up to 71% of cytotoxic T lymphocytes. Δ42PD-1⁺ T cells were tumor-infiltrating and correlated positively with HCC severity. Moreover, they were more exhausted than PD-1⁺ T cells by single T cell and functional analysis.

### Example 7—Anti-Tumor Effect of Anti-Δ42PD-1 Antibody CH101 in Murine Models

We have investigated HCC patients in three cohorts, including untreated, treated with Nivolumab and treated with isotype control. Peripheral blood mononuclear cells (PBMCs) from blood samples and tumor infiltrating lymphocytes (TILs) from tumor tissues were isolated for immunophenotyping. The functional significance of Δ42PD-1 was explored by single-cell RNA sequencing (scRNA-seq) analysis and validated by functional and mechanistic studies. The efficacy of Δ42PD-1 monoclonal antibody was determined in HCC humanized mouse models.

The detailed experimental procedures were as described in “MATERIALS AND METHODS” above.

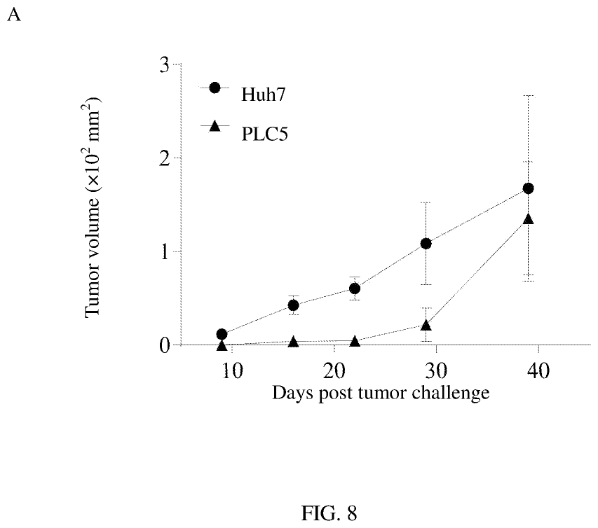

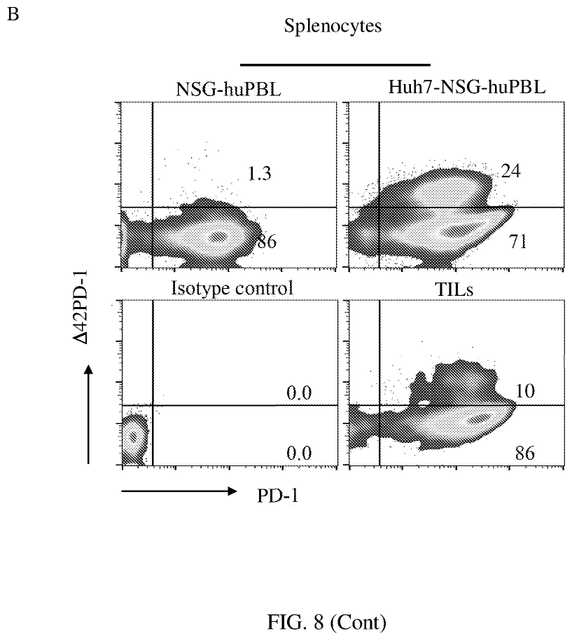

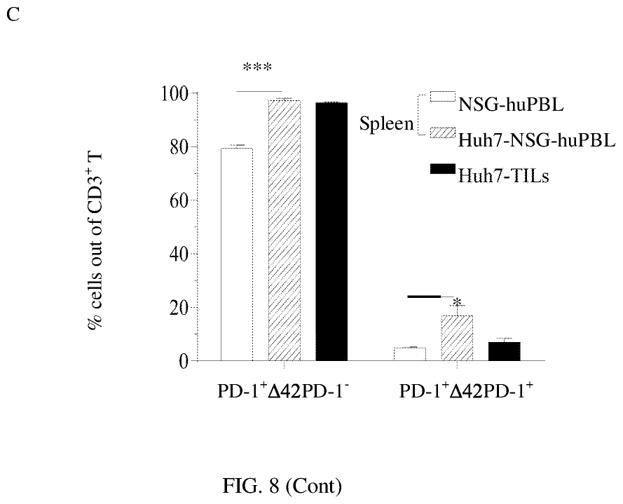

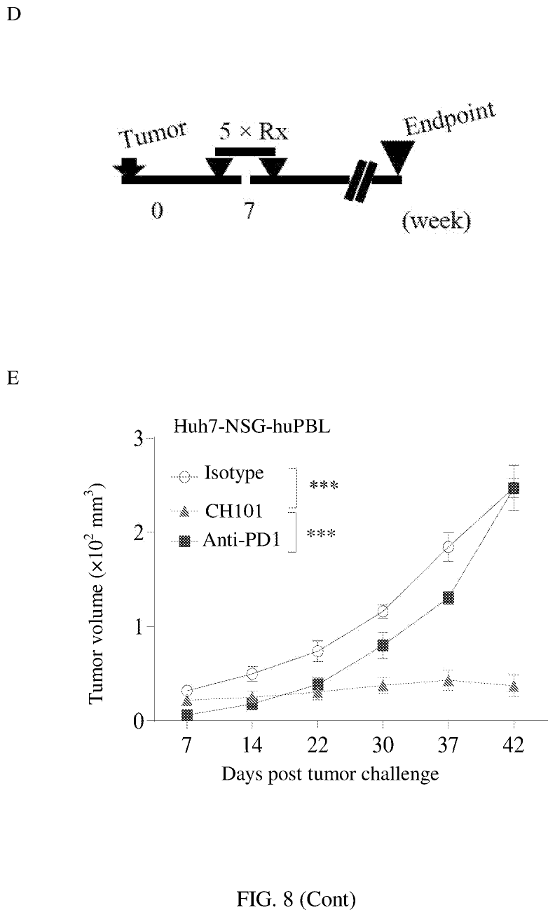

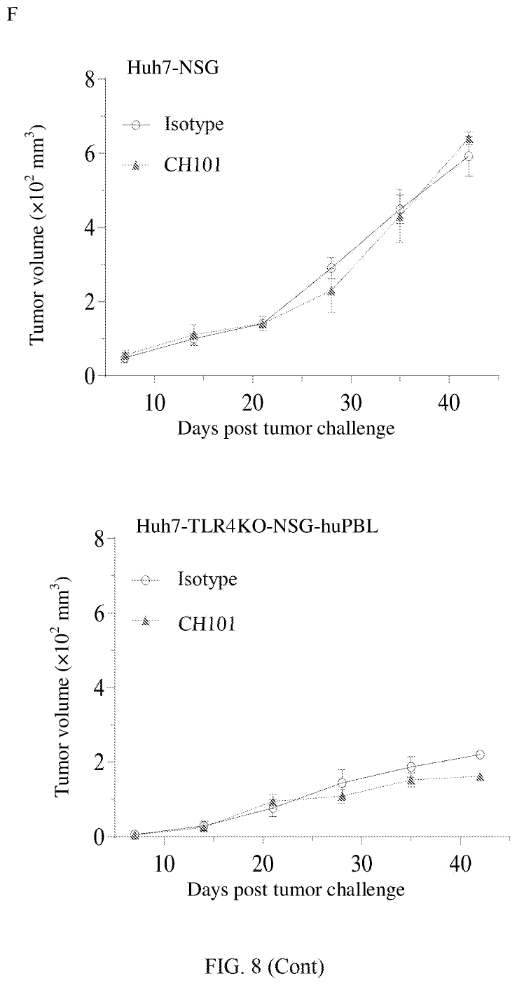

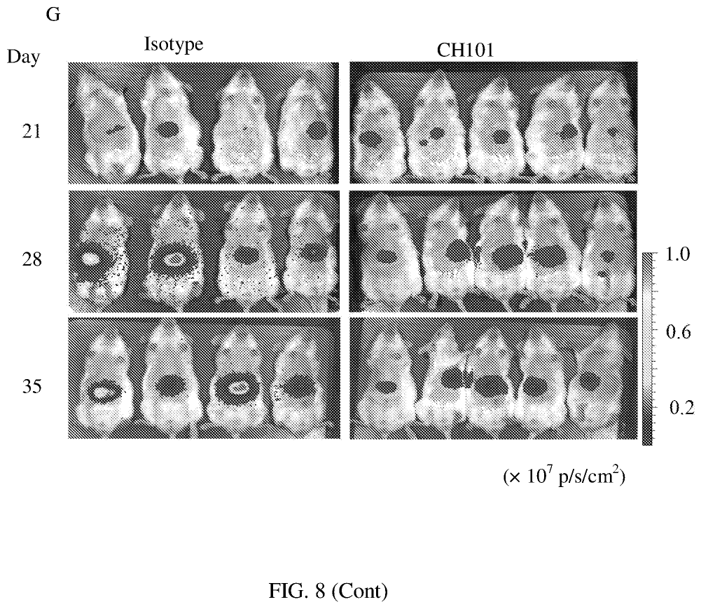

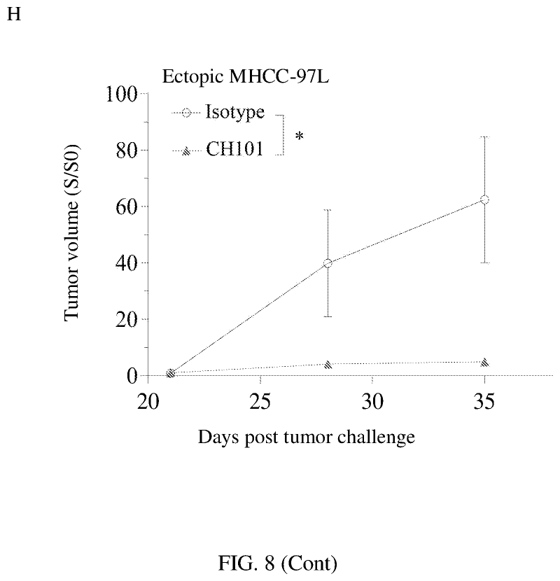

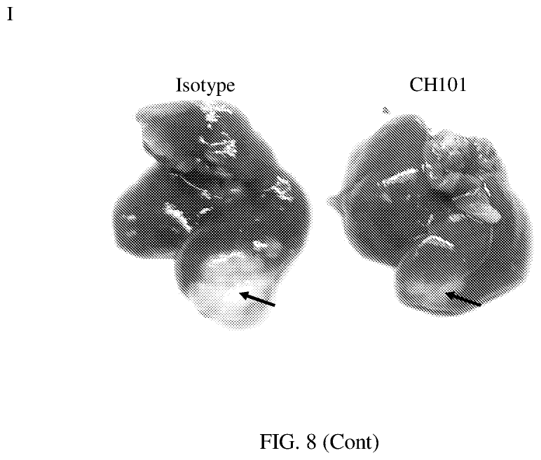

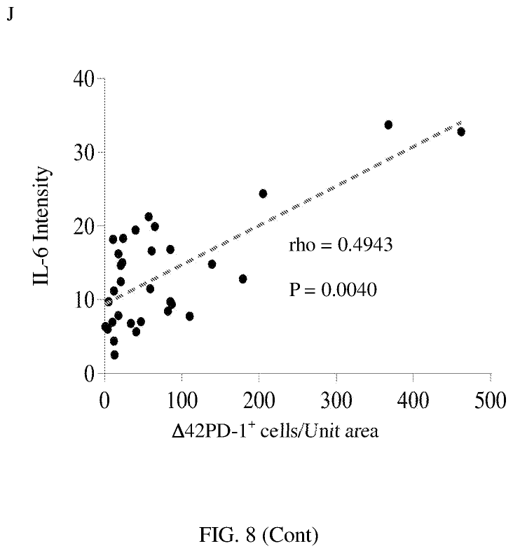

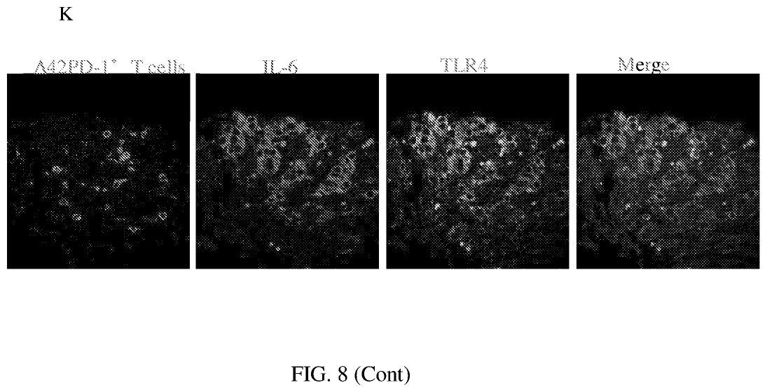

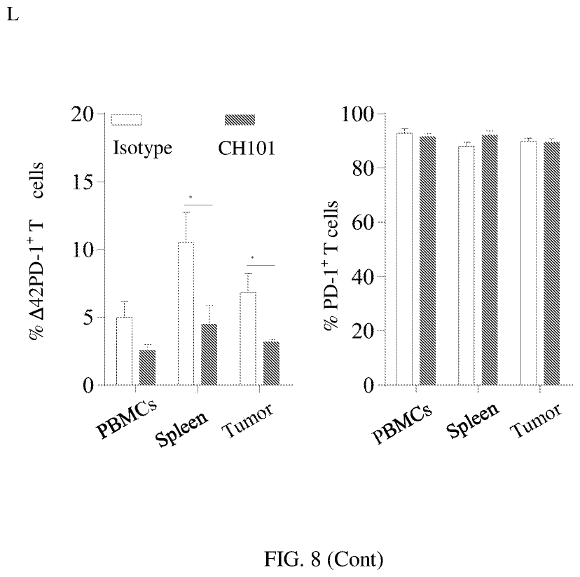

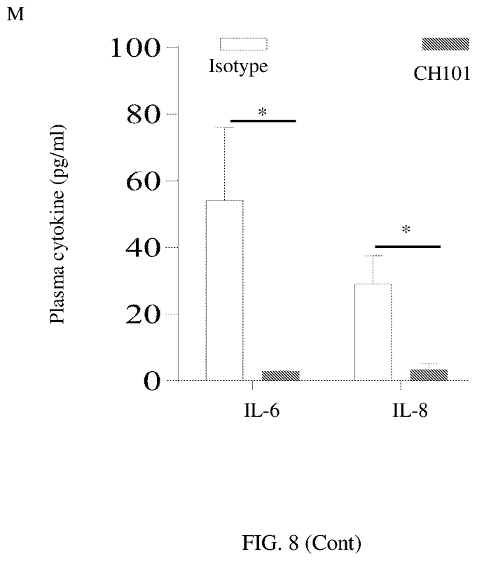

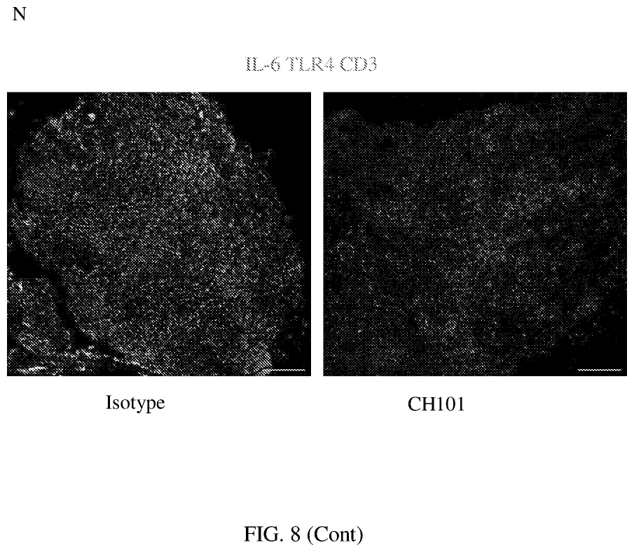

Frequencies of PD-1⁺Δ42PD1⁻ and PD-1⁺Δ42PD-1⁺ T cells out of total human CD45⁺CD3⁺ T cells in spleen or in TME were determined in NSG-huPBL and Huh7-NSG-huPBL mice (FIGS. 8B-8C). Tumor growth curve of Huh7-NSG-HuPBL mice after CH101, anti-PD-1 Nivolumab or isotype treatment was shown in FIG. 8E. Tumor growth curves for Huh7 in NSG mice or Huh7-TLR4KO in NSG-HuPBL mice was shown in FIG. 8F. Growth of orthotopic MHCC-97L tumors was measured by bioluminescent imaging and shown in FIG. 8H-8G. One representative liver image is shown at the endpoint (FIG. 8I). Spearman's correlations of Δ42PD-1⁺ T cells and IL-6 intensity in HCC patients were evaluated and shown in FIG. 8J. Representative images of one patient's tumor sample (×40) indicated the co-localization of Δ42PD-1⁺ TIL with IL-6-producing TLR4⁺ HCC cells (FIG. 8K). Quantification results of Δ42PD-1⁺ and PD-1⁺ T cells in PBMCs, spleens and tumors of Huh7-NSG-huPBL mice at the endpoint were shown in FIG. 8L. Quantification results of IL-6 and IL-8 in plasma at the endpoint were shown in FIG. 8M. Representative immunofluorescent staining (×20) is shown for tumor tissues derived from Huh7-NSG-huPBL mice, together with colour-coding for cell expression of IL-6, TLR4 and CD3, respectively (FIG. 8N).

The results indicate that HCC patients treated with anti-PD-1 ICB showed effective PD-1 blockade but increased frequencies of Δ42PD-1⁺ T cells over time especially in patients with progressive disease. Tumor-infiltrated Δ42PD-1⁺ T cells likely sustained HCC through TLR4-signaling for tumorigenesis. Anti-Δ42PD-1 antibody CH101, but not Nivolumab, inhibited tumor growth in murine HCC models.

Our findings not only revealed a mechanism underlying resistance to PD-1 ICB but also identified anti-Δ42PD-1 antibody CH101 for HCC immunotherapy.

### Example 8—Amplification and Construction of huCH34

In order to engineer the humanized CH34 antibodies and its variants, the variable heavy and light chains of murine CH34 were amplified from the selected hybridoma clone of CH34 using the similar method as described in EXAMPLE 1.

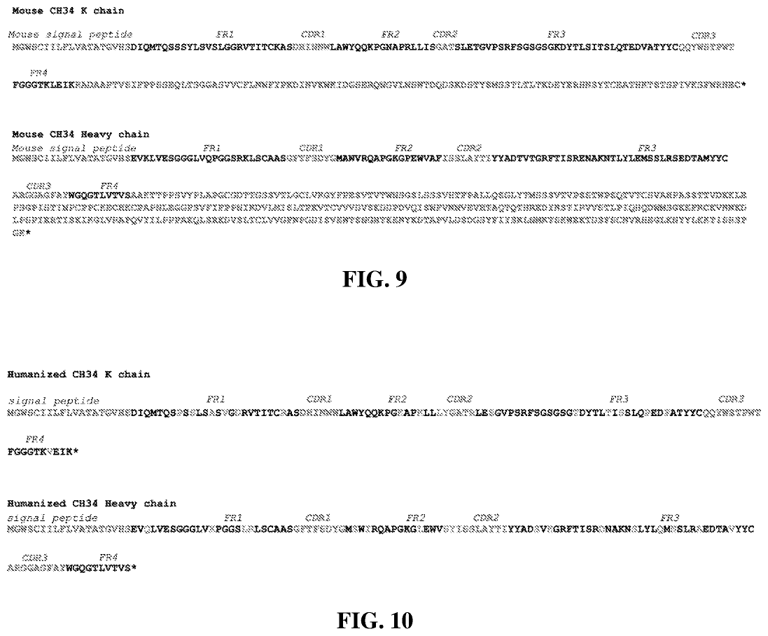

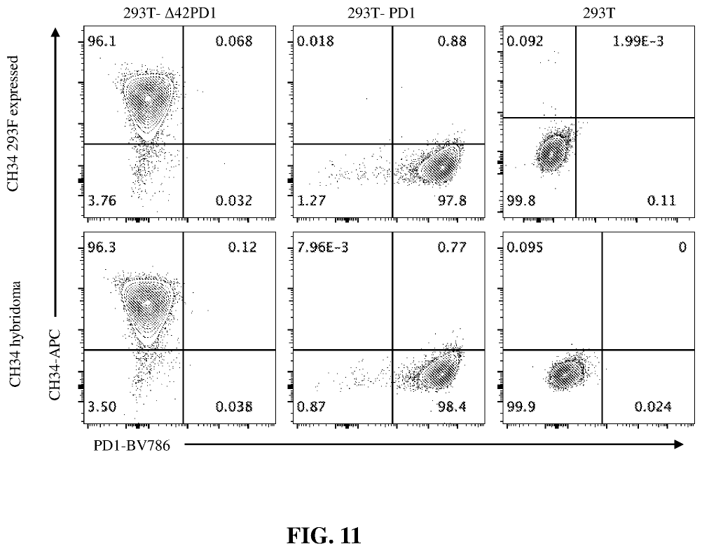

Amino acid sequences of heavy chain and light chain of mouse CH34 mAb were shown in FIG. 9. Amino acid sequences of heavy chain variable region (VH) and light chain variable region (VL) of humanized CH34 mAb were shown in FIG. 10.

### Example 9—Specificity of Anti-Δ42PD1 Antibody CH34 Binding to Δ42PD-1 Expressed on Human 293T Cells

To determine the specificity of anti-Δ42PD1 antibody CH34 and anti-PD1 antibody (EH12.2H7), 293T cells are transfected with a plasmid PCDH expressing Δ42PD-1, PD-1 or vector alone. Untransfected cells are included as controls. Two days after the transfection, the specificity of anti-Δ42PD1 antibody CH34 and anti-PD1 antibody EH12.2H7 is measured by analysis on PD-1 and Δ42PD1 expression.

The results were shown in FIG. 11. The results show that anti-Δ42PD1 antibody CH34 specifically binds to Δ42PD1 but not to PD-1 expressed on human 293T cells.

### Example 10—Specificity of Anti-Δ42PD1 Antibody CH34 Binding to Δ42PD-1 Expressed on Human B Cells

To test the specificity of anti-Δ42PD1 antibody CH34 as compared with the anti-PD1 antibody (EH12.2H7), RPMI8866 B cells are transfected with a plasmid PCDH expressing Δ42PD-1-GFP, PD-1-GFP or GFP along by Neon electroporation system. Untransfected RPMI8866 B cells are included as controls. Two days after the transfection, the transfection efficiency is determined by flow cytometry analysis on GFP (upper panel). The specificity of anti-Δ42PD1 antibody CH34 and anti-PD1 antibody EH12.2H7 is measured by analysis on PD-1 and Δ42PD1 expression (lower panel).

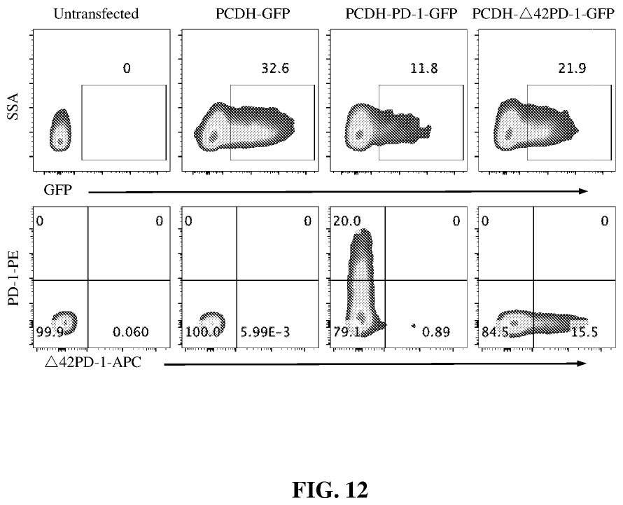

The results were shown in FIG. 12. The results show that anti-Δ42PD1 antibody CH34 specifically binds to Δ42PD1 but not to PD-1 expressed on human B cells.

### Example 11—Increased Δ42PD-1 Expression Accompanies B Cell Exhaustion in CHPs

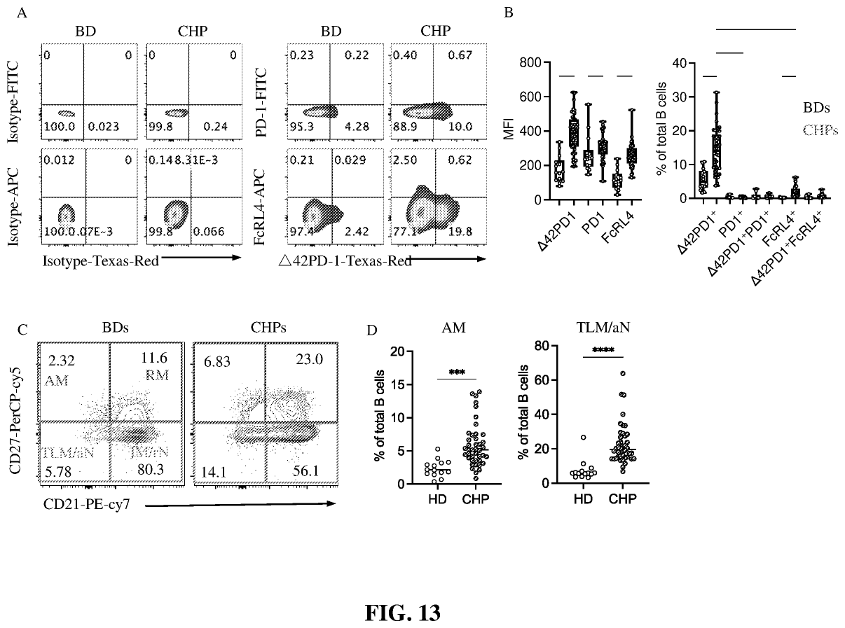

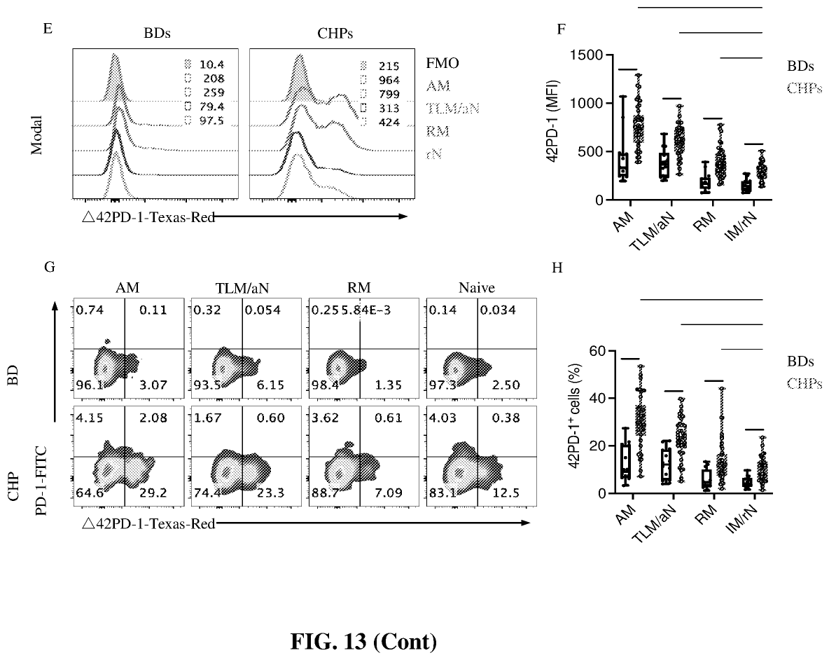

Molecular mechanisms that control the BCR-mediated human B cell activation and fate remain incompletely understood. Also, HIV-1 antibody responses are drained into exhausted B cells, yet the suppressor of B cell activation after B cell receptor (BCR) stimulation remains incompletely understood. To explore these mechanisms, Δ42PD-1 expression and B cell exhaustion were measured in BDs and chronic HIV-1 patients (CHPs). Representative flow cytometry analysis of CD19⁺ B cells derived from BDs and untreated CHPs was shown in FIG. 13A. FIG. 13B summarized data from FIG. 13A showing Median Fluorescence Intensity (MFI) of Δ42PD-1 and frequencies of Δ42PD-1⁺ B cells in BDs and CHPs. To identify CD21⁻CD27⁺ activated memory (AM), CD21⁻CD27⁺ resting memory (RM), CD27⁻CD21⁻ tissue like memory and anergic naïve B (TLM/aN), and CD27⁻CD21⁺ resting naïve plus intermediated memory B cells (IM/rN), gating on CD21/CD27-stained B cells of a representative BD and CHP was performed and the results were shown in FIG. 13C. FIG. 13D summarized data from FIG. 13C showing increased frequencies of AM and TLM/aN B cells in CHPs in comparison with BDs. Δ42PD-1 expression on AM, TLM/aN, RM and IM/rN B cells of a representative BD and CHP was shown in FIG. 13E. FIG. 13F summarized data from FIG. 13E showing MFI of Δ42PD-1 on B cell subsets in BDs and CHPs. Representative flow cytometry analysis of PD-1⁺ and Δ42PD-1⁺ AM, TLM/aN, RM and IM/rN B cells derived from BD and CHP was shown in FIG. 13G. FIG. 13H summarized data from FIG. 13G showing frequencies of Δ42PD-1⁺ AM, TLM/aN, RM and IM/rN B cells in BDs and CHPs.

These results indicate that Δ42PD-1 is predominantly up-regulated on activated memory, anergic naïve and tissue like memory B cells compared with PD-1 and FcRL4 in CHPs.

### Example 12—Anti-Δ42PD-1 Antibody Reduces SHP1 Recruitment, Enhances AKT1/FOXO1 Pathway and B Cell Proliferation

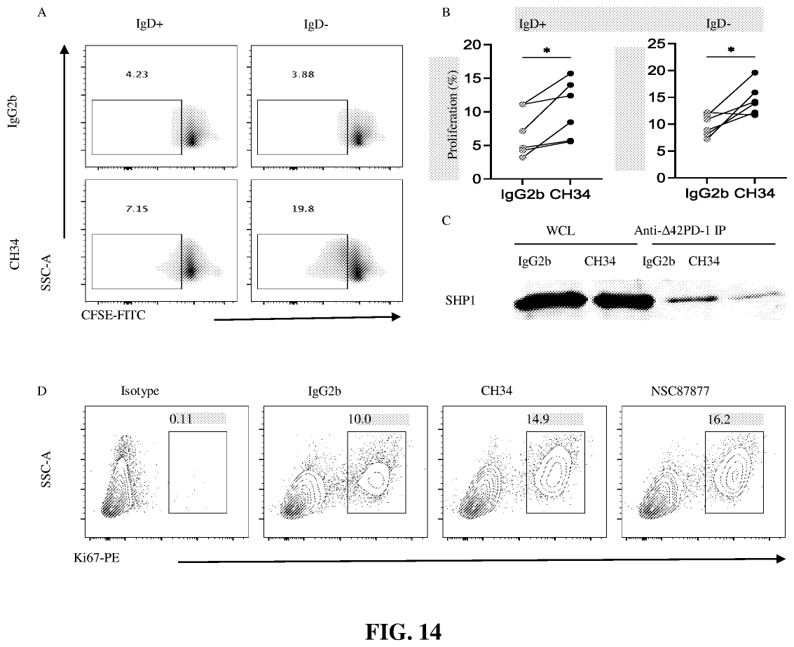

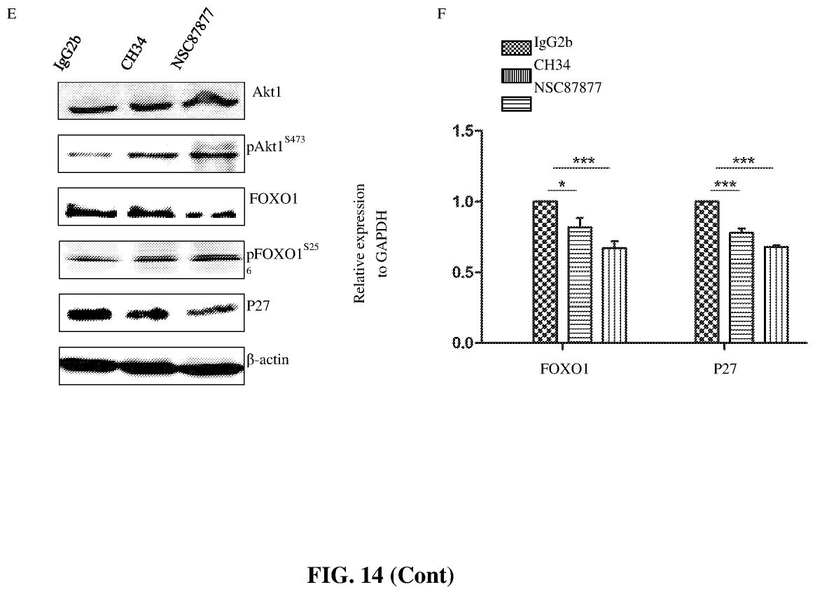

To determine whether CH34 enhances B cell proliferation upon BCR-stimulation, flow cytometry was performed. Primary B cells from BDs (n=6) are prelabelled with FITC-CFSE and stimulated with α-IgM/G F(ab′)2 (10 μg/ml) plus msIgG2b control antibody or CH34. Cells are analyzed by flow cytometry at day 5. The results were shown in FIGS. 14A-14B.

To determine whether CH34 reduces SHP1 recruitment by Δ42PD-1, Primary B cells from BDs are stimulated with α-IgM/G F(ab′)2 (10 μg/ml) plus msIgG2b control antibody or CH34, and lysed at day 2 for immunoprecipitation and western blot using antibodies against Δ42PD-1 and SHP1, respectively. The results were shown in FIG. 14C.

To determine the affect of CH34 treatment on Ki67⁺ B cell proliferation, on phoph-AKT1-S473 and phosph-FOXO1-S256 level, and P27 protein expression, and on FOXO1 and P27 mRNA expression in B cells, primary B cells were cultured in complete RPMI1640 medium with α-IgM/G F(ab′)2 (10 μg/ml) and msIgG2b control antibody (10 g/ml) or CH34 (10 μg/ml), or SHP1 inhibitor NSC87877(10 uM). 48 h later, cells were stained for flow cytometry analysis or lysed for western blotting detection of AKT1, FOXO1 and P27, or qPCR for mRNA of FOXO1 and P27. The results were shown in FIGS. 14D-14F.

The results indicate that CH34 enhances B cell proliferation upon BCR-stimulation, CH34 reduces SHP1 recruitment by Δ42PD-1, CH34 treatment expanded Ki67⁺ B cell population, CH34 treatment increases phoph-AKT1-S473 and phosph-FOXO1-S256 level, and decreases P27 protein expression, and CH34 treatment increases FOXO1 and P27 mRNA in B cells.

### Example 13—CH34 Enhances B Cell Proliferation after BCR-Stimulation in HIV Patients

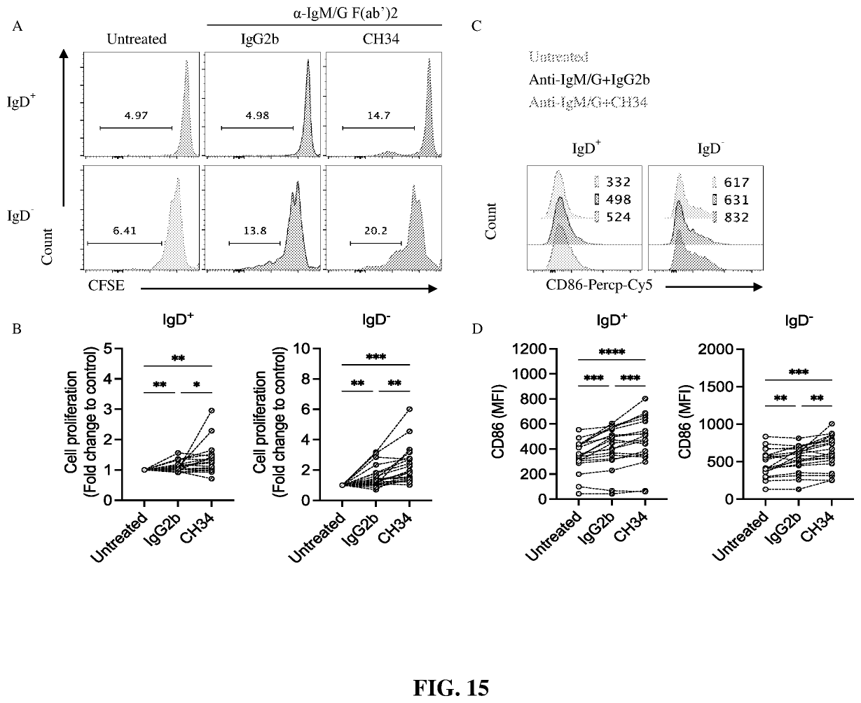

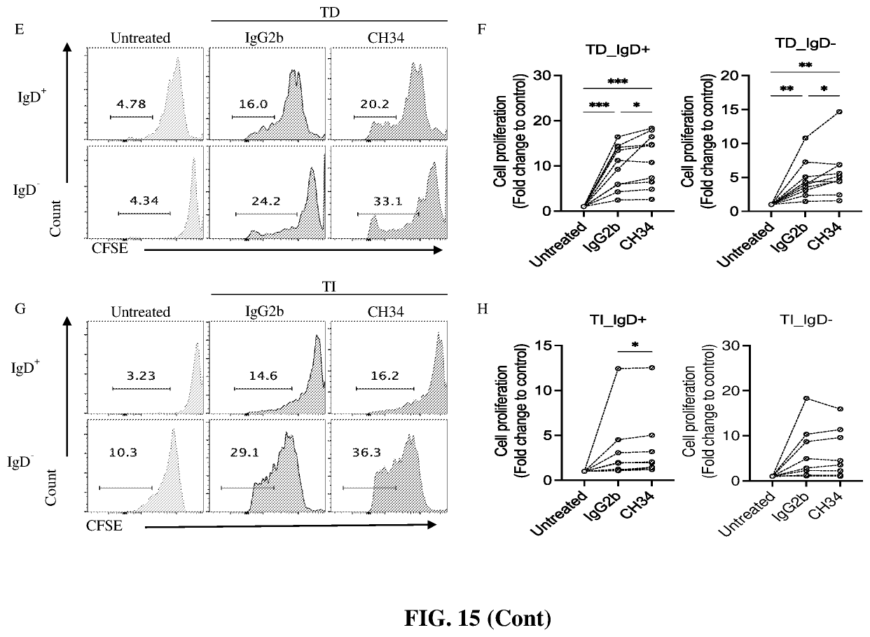

To determine the affect of CH34 on B cell proliferation after BCR-stimulation in HIV patients, flow cytometry was performed. IgD⁺ and IgD⁻ B cells isolated from PBMC of CHPs are prelabelled with CFSE and cultured in RPMI1640 medium with α-IgM/G F(ab′)2 (10 μg/ml), TD or TI stimulation plus CH34 (10 μg/ml) or msIgG2b control (10 g/ml) for 5 days, and analyzed by flow cytometry. Statistical significance is determined using ordinary one-way ANOVA and the two-stage linear step-up procedure of Benjamini, Krieger and Yekutieli, with a single pooled variance. The results were shown in FIGS. 15A-15H.

The results indicate that CH34 increases proliferative response of naïve (IgD⁺) and memory (IgD⁻) B cell after BCR-stimulation (FIGS. 15A-15B), increases CD86 expression in naïve and memory B cells from CHPs upon BCR stimulation (FIGS. 15C-15D), increases proliferative response of B cells from CHPs after TD stimulation (FIGS. 15E-15F), and increases proliferative response of naïve B cells from CHPs after TI stimulation.

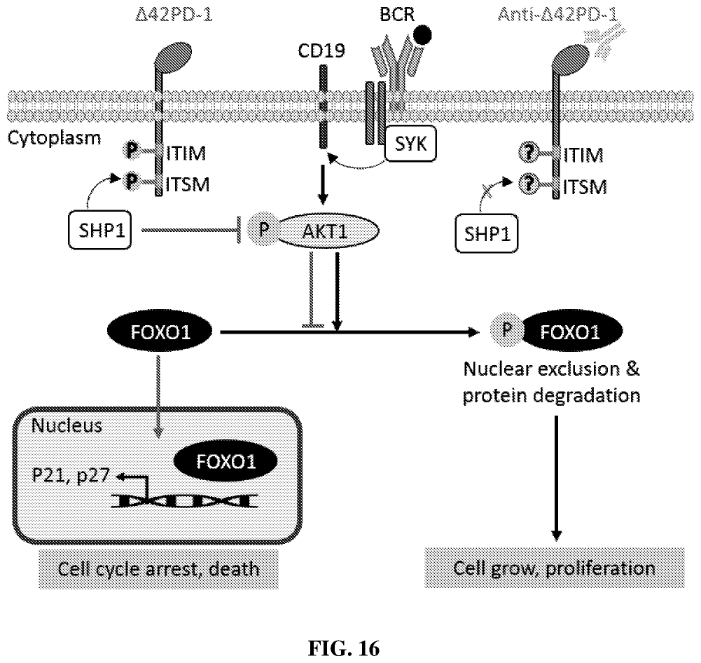

Our results suggest that BCR-stimulation up-regulates Δ42PD-1, resulting in B cell exhaustion, cell cycle arrest and death. Mechanistically, Δ42PD-1 recruits Src homology region 2 domain containing phosphatase 1 (SHP1) via its intracellular immunoreceptor tyrosine-based switch motif (ITSM). SHP1 then binds and inhibits AKT1 activation and thereby suppresses the AKT1/FOXO1 pathway. Δ42PD-1-specific antibody CH34, however, reduces the SHP1 recruitment, increasing the AKT1/FOXO1 activation and proliferation of B cells derived from CHPs (FIG. 16).

Our findings demonstrate that Δ42PD-1 is a previously unrecognized suppressor of BCR-mediated B cell activation, which may serve as a potential immunotherapeutic target for restoring B cell functionality.

### SEQUENCES

Protein
Artificial Sequence: CH101 VH amino acid
sequence
SEQ ID NO: 1
EVQLEESGPDLVKPSQSLSLTCTVTGYSITSGFAWHWLRQFPGNRLEWMG
YIHYSGSTNYNPSLKSRISITRDTSKNQFFLQLNSVTTEDTATFYCARSD
FWGQGTFVTVSS
Protein
Artificial Sequence: CH101 VK amino acid
sequence
SEQ ID NO: 2
DVVMTQTPLNLSVTFGQPVSISCTSSQSLLDRDGETFMNWLFQRPGQSPK
RLIYLVSKLDFGVPDRFTGSGSGTDFTLKISRVEAEDLGIYYCWQGTHFP
LTFGGGTKLEIKRGQAG
Protein
Artificial Sequence: hufrCH101 VH amino
acid sequence
SEQ ID NO: 3
QVQLQESGPGLVKPSETLSLTCTVSGYSITSGFAWHWIRQPPGKGLEWIG
YIHYSGSTNYNPSLKSRVTISVDTSKNQFSLKLSSVTAADTAVYYCARSD
FWGQGTLVTVSS
Protein
Artificial Sequence: hufrCH101 VK amino
acid sequence
SEQ ID NO: 4
DVVMTQSPLSLPVTLGQPASISCTSSQSLLDRDGETFMNWFQQRPGQSPR
RLIYLVSKLDFGVPDRFSGSGSGTDFTLKISRVEAEDVGVYYCWQGTHFP
LTFGGGTKVEIKRGQAG
DNA
Artificial Sequence: CH101 VH nucleic
acid sequence
SEQ ID NO: 5
GAGGTGCAGCTCGAGGAGTCAGGACCTGACCTGGTGAAACCTTCTCAGTC
ACTTTCACTCACCTGCACTGTCACTGGCTATTCCATCACCAGTGGTTTTG
CCTGGCACTGGCTCCGGCAGTTTCCAGGAAACAGACTGGAATGGATGGGC
TACATACACTATAGTGGAAGCACTAACTACAACCCATCTCTCAAAAGTCG
AATCTCTATCACTCGAGACACATCCAAGAATCAGTTCTTCCTGCAGTTGA
ATTCTGTGACTACTGAGGACACAGCCACTTTTTACTGTGCAAGGTCTGAC
TTCTGGGGCCAAGGCACCTTTGTCACAGTCTCCTCA
DNA
Artificial Sequence: CH101 VK nucleic
acid sequence
SEQ ID NO: 6
GATGTTGTGATGACCCAGACTCCACTCAATTTGTCGGTCACCTTTGGACA
ACCAGTCTCCATCTCTTGCACGTCTAGTCAGAGCCTCTTAGATCGTGATG
GAGAGACATTCATGAATTGGTTGTTCCAGCGGCCAGGCCAGTCTCCAAAG
CGCCTAATCTATCTGGTGTCTAAACTGGACTTTGGAGTCCCTGACAGGTT
CACTGGCAGTGGATCAGGGACAGATTTCACACTGAAAATCAGCAGAGTGG
AGGCTGAGGATTTGGGAATTTATTATTGCTGGCAAGGTACACATTTTCCT
CTCACGTTCGGTGGTGGGACCAAGCTGGAAATAAAACGCGGCCAGGCCGG
C
DNA
Artificial Sequence: hufrCH101 VH
nucleic acid sequence
SEQ ID NO: 7
CAGGTTCAGCTCCAGGAGTCAGGACCCGGTCTGGTGAAACCTTCAGAGAC
ACTGAGTCTGACATGTACGGTAAGTGGGTACTCCATTACTTCCGGTTTTG
CTTGGCATTGGATACGGCAACCGCCTGGGAAGGGGTTGGAGTGGATAGGT
TATATTCATTATAGCGGGTCAACAAATTATAACCCATCACTCAAGTCCCG
CGTCACGATAAGCGTTGATACCTCCAAGAATCAGTTCAGCTTGAAATTGT
CAAGCGTAACAGCCGCCGATACCGCAGTGTATTATTGCGCAAGGAGTGAC
TTTTGGGGCCAAGGTACGCTTGTCACCGTAAGTTCC
DNA
Artificial Sequence: hufrCH101 VK
nucleic acid sequence
SEQ ID NO: 8
GACGTAGTCATGACCCAGTCCCCACTGTCATTGCCGGTAACGCTTGGACA
GCCTGCTAGTATCTCATGCACATCCAGCCAATCCCTCCTTGACCGCGACG
GTGAGACGTTTATGAATTGGTTCCAGCAGAGACCAGGACAGTCACCGAGG
AGACTTATTTACCTTGTTTCAAAGCTCGACTTCGGTGTCCCCGACAGATT
TAGCGGTAGTGGTTCTGGGACAGATTTCACCCTTAAAATCAGTAGGGTCG
AAGCGGAGGACGTGGGTGTCTACTATTGTTGGCAAGGCACGCACTTTCCA
TTGACCTTTGGCGGGGGGACAAAGGTGGAAATTAAACGGGGCCAGGCGGG
G
Protein
Homo sapiens: Amino acid sequence of
germline variable heavy chain for CDR
grafting (GenBank: BAA75026.1, Accession
No .: AB019437.1)
SEQ ID NO: 9
QVQLQESGPGLVKPSETLSLTCTVSGGSVSSGGYYWSWIRQPPGKGLEWI
GYIYYSGSTNYNPSLKSRVTISVDTSKNQFSLKLSSVTAADTAVYYCAR
Protein
Homo sapiens: Amino acid sequence of
germline variable kappa chain for CDR
grafting (GeneBank: BAC01687.1, Accession
No .: AB064059.1)
SEQ ID NO: 10
DVVMTQSPLSLPVTLGQPASISCRSSQSLVYSDGNTYLNWFQQRPGQSPR
RLIYKVSNRDSGVPDRFSGSGSGTDFTLKISRVEAEDVGVYYCMQGTHWP
P
Protein
Homo sapiens: Amino acid of J region of
human heavy chain (IGHJ4*01)
SEQ ID NO: 11
YFDYWGQGTLVTVSS
Protein
Homo sapiens: Amino acid of J region of
human kappa chain (IGKJ4*01)
SEQ ID NO: 12
TFGGGTKVEIK
DNA
Homo sapiens: Nucleic acid sequence of
germline variable heavy chain for CDR
grafting (GenBank: BAA75026.1, Accession
No .: AB019437.1)
SEQ ID NO: 13
CAGGTGCAGCTGCAGGAGAGCGGCCCCGGCCTGGTGAAGCCCAGCGAGAC
CCTGAGCCTGACCTGCACCGTGAGCGGCGGCAGCGTGAGCAGCGGCGGCT
ACTACTGGAGCTGGATCAGGCAGCCCCCCGGCAAGGGCCTGGAGTGGATC
GGCTACATCTACTACAGCGGCAGCACCAACTACAACCCCAGCCTGAAGAG
CAGGGTGACCATCAGCGTGGACACCAGCAAGAACCAGTTCAGCCTGAAGC
TGAGCAGCGTGACCGCCGCCGACACCGCCGTGTACTACTGCGCCAGG
DNA
Homo sapiens: Nucleic acid sequence of
germline variable kappa chain for CDR
grafting (GeneBank: BAC01687.1, Accession
No .: AB064059.1)
SEQ ID NO: 14
GACGTGGTGATGACCCAGAGCCCCCTGAGCCTGCCCGTGACCCTGGGCCA
GCCCGCCAGCATCAGCTGCAGGAGCAGCCAGAGCCTGGTGTACAGCGACG
GCAACACCTACCTGAACTGGTTCCAGCAGAGGCCCGGCCAGAGCCCCAGG
AGGCTGATCTACAAGGTGAGCAACAGGGACAGCGGCGTGCCCGACAGGTT
CAGCGGCAGCGGCAGCGGCACCGACTTCACCCTGAAGATCAGCAGGGTGG
AGGCCGAGGACGTGGGCGTGTACTACTGCATGCAGGGCACCCACTGGCCC
CCC
DNA
Homo sapiens: Nucleic acid of J region
of human heavy chain (IGHJ4*01)
SEQ ID NO: 15
TACTTTGACTACTGGGGCCAAGGAACCCTGGTCACCGTCTCCTCA
DNA
Homo sapiens: Nucleic acid of J region
of human kappa chain (IGKJ4*01)
SEQ ID NO: 16
ACTTTCGGCGGAGGGACCAAGGTGGAGATCAAA
DNA
Homo sapiens: human kappa chain constant
fragment nucleic acid sequence
SEQ ID NO: 17
CGTACGGTGGCTGCACCATCTGTCTTCATCTTCCCGCCATCTGATGAGCA
GTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGT
AACTCCCAGGAGAGTGTCACAGAGCAGGACAGCAAGGACAGCACCTACAG
CCTCAGCAGCACCCTGACGCTGAGCAAAGCAGACTACGAGAAACACAAAG
TCTACGCCTGCGAAGTCACCCATCAGGGCCTGAGCTCGCCCGTCACAAAG
AGCTTCAACAGGGGAGAGTGT
Protein:
Homo sapiens: human kappa chain constant
fragment amino acid sequence
SEQ ID NO: 18
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
Protein
Homo sapiens: human heavy chain constant
fragment amino acid sequence (IgG1)
SEQ ID NO: 19
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
DNA
Homo sapiens: human heavy chain constant
fragment nucleic acid sequence (IgG1)
SEQ ID NO: 20
GCGTCGACCAAGGGCCCATCGGTCTTCCCCCTGGCACCCTCCTCCAAGAG
CACCTCTGGGGGCACAGCGGCCCTGGGCTGCCTGGTCAAGGACTACTTCC
CCGAACCTGTGACGGTCTCGTGGAACTCAGGCGCCCTGACCAGCGGCGTG
CACACCTTCCCGGCTGTCCTACAGTCCTCAGGACTCTACTCCCTCAGCAG
CGTGGTGACCGTGCCCTCCAGCAGCTTGGGCACCCAGACCTACATCTGCA
ACGTGAATCACAAGCCCAGCAACACCAAGGTGGACAAGAGAGTTGAGCCC
AAATCTTGTGACAAAACTCACACATGCCCACCGTGCCCAGCACCTGAACT
CCTGGGGGGACCGTCAGTCTTCCTCTTCCCCCCAAAACCCAAGGACACCC
TCATGATCTCCCGGACCCCTGAGGTCACATGCGTGGTGGTGGACGTGAGC
CACGAAGACCCTGAGGTCAAGTTCAACTGGTACGTGGACGGCGTGGAGGT
GCATAATGCCAAGACAAAGCCGCGGGAGGAGCAGTACAACAGCACGTACC
GTGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAA
AACCATCTCCAAAGCCAAAGGGCAGCCCCGAGAACCACAGGTGTACACCC
TGCCCCCATCCCGGGAGGAGATGACCAAGAACCAGGTCAGCCTGACCTGC
CTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCG
ACGGCTCCTTCTTCCTCTATAGCAAGCTCACCGTGGACAAGAGCAGGTGG
CAGCAGGGGAACGTCTTCTCATGCTCCGTGATGCATGAGGCTCTGCACAA
CCACTACACGCAGAAGAGCCTCTCCCTGTCCCCGGGTAAA
Protein
Homo sapiens: human heavy chain constant
fragment amino acid sequence (IgG4)
SEQ ID NO: 21
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
DNA
Homo sapiens: human heavy chain constant
fragment nucleic acid sequence (IgG4)
SEQ ID NO: 22
GCGTCGACCAAGGGACCAAGCGTATTCCCGCTTGCGCCATGTAGCAGATC
AACAAGTGAGTCCACGGCGGCCCTTGGGTGCTTGGTAAAGGATTACTTTC
CTGAGCCAGTAACCGTGTCTTGGAATTCAGGAGCTCTCACGTCAGGGGTA
CACACCTTCCCTGCCGTCCTTCAATCTTCTGGCCTCTATAGCCTCTCAAG
CGTCGTTACTGTGCCATCCTCCTCACTCGGAACTAAAACATATACTTGCA
ACGTAGACCACAAGCCCTCAAATACAAAAGTTGACAAACGAGTTGAATCT
AAGTACGGCCCGCCATGTCCGCCATGTCCAGCCCCCGAATTCTTGGGGGG
ACCATCCGTATTTCTTTTCCCGCCGAAACCCAAAGACACCTTGATGATAA
GCCGAACACCAGAGGTTACATGTGTCGTTGTTGATGTCTCTCAGGAAGAT
CCTGAGGTCCAGTTTAACTGGTACGTAGATGGGGTGGAAGTTCACAACGC
AAAAACCAAGCCGAGAGAGGAACAGTTCAACTCAACCTATCGGGTTGTTT
CCGTTCTCACAGTTCTGCATCAGGACTGGCTTAATGGAAAAGAATACAAG
TGTAAAGTGTCAAATAAGGGCCTCCCATCAAGCATTGAAAAGACCATATC
TAAGGCCAAAGGCCAACCTCGCGAGCCTCAAGTCTACACCTTGCCTCCTA
GCCAAGAAGAAATGACAAAGAACCAAGTATCTCTCACATGTCTTGTTAAA
GGGTTCTATCCAAGCGATATCGCTGTTGAGTGGGAAAGCAATGGCCAACC
TGAAAATAACTACAAAACCACTCCTCCCGTGCTTGACTCAGATGGTTCTT
TCTTCCTCTATTCACGATTGACTGTGGACAAAAGTCGCTGGCAGGAAGGG
AACGTTTTCTCCTGTAGCGTGATGCACGAGGCGCTCCACAATCATTATAC
CCAAAAGTCACTCTCTCTTAGCCTTGGCAAG
Protein
Artificial Sequence: CH101 VH CDR1
(Kabat numbering system)
SEQ ID NO: 23
GYSITSGF
Protein
Artificial Sequence: CH101 VH CDR2
(Kabat numbering system)
SEQ ID NO: 24
HYSGS
Protein
Artificial Sequence: CH101 VH CDR3
(Kabat numbering system)
SEQ ID NO: 25
SDF
Protein
Artificial Sequence: CH101 VL CDR1
(Kabat numbering system)
SEQ ID NO: 26
TSSQSLLDRDGETFMN
Protein
Artificial Sequence: CH101 VL CDR2
(Kabat numbering system)
SEQ ID NO: 27
LVSKLDF
Protein
Artificial Sequence: CH101 VH CDR3
(Kabat numbering system)
SEQ ID NO: 28
WQGTHFPL
Protein
Artificial Sequence: CH34 VH CDR1
(Kabat numbering system)
SEQ ID NO: 29
GFTFSDYG
Protein
Artificial Sequence: CH34 VH CDR2
(Kabat numbering system)
SEQ ID NO: 30
ISSLAYTI
Protein
Artificial Sequence: CH34 VH CDR3
(Kabat numbering system)
SEQ ID NO: 31
ARGGAGFAY
Protein
Artificial Sequence: CH34 VL CDR1
(Kabat numbering system)
SEQ ID NO: 32
DHINNW
Protein
Artificial Sequence: CH34 VL CDR2
(Kabat numbering system)
SEQ ID NO: 33
GAT
Protein
Artificial Sequence: CH34 VL CDR3
(Kabat numbering system)
SEQ ID NO: 34
QQYWSTPWT
Protein
Artificial Sequence: Mouse CH34 VH
SEQ ID NO: 35
EVKLVESGGGLVQPGGSRKLSCAASGFTFSDYGMAWVRQAPGKGPEWVAF
ISSLAYTIYYADTVTGRFTISRENAKNTLYLEMSSLRSEDTAMYYCARGG
AGFAYWGQGTLVTVS
Protein
Artificial Sequence: Mouse CH34 VL
SEQ ID NO: 36
DIQMTQSSSYLSVSLGGRVTITCKASDHINNWLAWYQQKPGNAPRLLISG
ATSLETGVPSRFSGSGSGKDYTLSITSLQTEDVATYYCQQYWSTPWTFGG
GTKLEIK
Protein
Artificial Sequence: Humanized CH34 VH
SEQ ID NO: 37
EVQLVESGGGLVKPGGSLRLSCAASGFTFSDYGMSWIRQAPGKGLEWVSY
ISSLAYTIYYADSVKGRFTISRDNAKNSLYLQMNSLRAEDTAVYYCARGG
AGFAYWGQGTLVTVS
Protein
Artificial Sequence: Humanized CH34 VL
SEQ ID NO: 38
DIQMTQSPSSLSASVGDRVTITCRASDHINNWLAWYQQKPGKAPKLLLYG
ATRLESGVPSRFSGSGSGTDYTLTISSLQPEDFATYYCQQYWSTPWTFGG
GTKVEIK
Protein
Artificial Sequence: Signal peptide
SEQ ID NO: 39
MGWSCIILFLVATATGVHS
Protein
Artificial Sequence: Mouse CH34 heavy chain
SEQ ID NO: 40
EVKLVESGGGLVQPGGSRKLSCAASGFTFSDYGMAWVRQAPGKGPEWVAF
ISSLAYTIYYADTVTGRFTISRENAKNTLYLEMSSLRSEDTAMYYCARGG
AGFAYWGQGTLVTVSAAKTTPPSVYPLAPGCGDTTGSSVTLGCLVKGYFP
ESVTVTWNSGSLSSSVHTFPALLQSGLYTMSSSVTVPSSTWPSQTVTCSV
AHPASSTTVDKKLEPSGPISTINPCPPCKECHKCPAPNLEGGPSVFIFPP
NIKDVLMISLTPKVTCVVVDVSEDDPDVQISWFVNNVEVHTAQTQTHRED
YNSTIRVVSTLPIQHQDWMSGKEFKCKVNNKDLPSPIERTISKIKGLVRA
PQVYILPPPAEQLSRKDVSLTCLVVGFNPGDISVEWTSNGHTEENYKDTA
PVLDSDGSYFIYSKLNMKTSKWEKTDSFSCNVRHEGLKNYYLKKTISRSP
GK
Protein
Artificial Sequence: Mouse CH34 kappa chain
SEQ ID NO: 41
DIQMTQSSSYLSVSLGGRVTITCKASDHINNWLAWYQQKPGNAPRLLISG
ATSLETGVPSRFSGSGSGKDYTLSITSLQTEDVATYYCQQYWSTPWTFGG
GTKLEIKRADAAPTVSIFPPSSEQLTSGGASVVCFLNNFYPKDINVKWKI
DGSERQNGVLNSWTDQDSKDSTYSMSSTLTLTKDEYERHNSYTCEATHKT
STSPIVKSFNRNEC

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A humanized Δ42PD1-specific monoclonal antibody (mAb).

2. The humanized Δ42PD1-specific mAb of claim 1, wherein a variable heavy chain comprises at least 80% sequence identity to SEQ ID NO: 3 and a variable light chain comprises at least 80% sequence identity to SEQ ID NO: 4.

3. The humanized Δ42PD1-specific mAb of claim 1, wherein constant regions of heavy and light chains are human IgG1 constant heavy chain fragment, according to SEQ ID NO: 19 or a sequence having at least 80% identity to SEQ ID NO: 19, and human IgG1 light chain fragment, according to SEQ ID NO: 18 or a sequence having at least 80% identity to SEQ ID NO: 18.

4. A composition comprising the humanized Δ42PD1-specific mAb of claim 1, optionally, further comprising a pharmaceutically acceptable carrier and/or excipient.

5. (canceled)

6. A nucleic acid molecule encoding the humanized Δ42PD1-specific mAb of claim 1.

7. The nucleic acid molecule of claim 6, wherein; (a) the nucleic acid molecule is a plasmid; (b) the nucleic acid sequence encoding a variable heavy chain of the humanized □42PD1-specific mAb comprises at least 80% sequence identity to SEQ ID NO: 7 and the nucleic acid sequence encoding a variable light chain of the humanized □42PD1-specific mAb comprises at least 80% sequence identity to SEQ ID NO: 8; and/or (c) nucleic acid sequences encoding constant regions of the heavy and light chains are nucleic acid sequences encoding human IgG1 constant heavy chain fragment, according to SEQ ID NO: 20 or a sequence having at least 80% sequence identity to SEQ ID NO: 20, and human constant light chain fragment, according to SEQ ID NO: 17 or a sequence having at least 80% sequence identity to SEQ ID NO: 17.

8. (canceled)

9. (canceled)

10. A method of inhibiting cancer cell proliferation in a subject, comprising administering to the subject a humanized Δ42PD1-specific monoclonal antibody (mAb).

11. The method of claim 10, wherein: (a) a variable heavy chain of the humanized Δ42PD1-specific mAb comprises at least 80% sequence identity to SEQ ID NO: 3 and a variable light chain of the humanized Δ42PD1-specific monoclonal antibody comprises at least 80% sequence identity to SEQ ID NO: 4; and/or (b) constant regions of heavy and light chains of the humanized Δ42PD1-specific monoclonal antibody are human IgG1 constant heavy and light chain fragments; optionally, wherein the human IgG1 constant heavy chain has a sequence according to SEQ ID NO: 19 or a sequence having at least 80% identity to SEQ ID NO: 19 and the human IgG1 light chain fragment has a sequence according to SEQ ID NO: 18 or a sequence having at least 80% identity to SEQ ID NO: 18.

12. (canceled)

13. (canceled)

14. A Δ42PD1-specific antibody or an antigen binding fragment thereof, comprising a heavy chain variable region (VH) and a light chain variable region (VL), wherein
(i) the VH comprises HCDRs 1-3 having the amino acid sequences as shown in SEQ ID NOs: 23-25 respectively, and the VL comprises HCDRs 1-3 having the amino acid sequences as shown in SEQ ID NOs: 26-28 respectively; or
(ii) the VH comprises HCDRs 1-3 having the amino acid sequences as shown in SEQ ID NOs: 29-31 respectively, and the VL comprises LCDRs 1-3 having the amino acid sequences as shown in SEQ ID NOs: 32-34 respectively.

15. The antibody or the antigen binding fragment thereof of claim 14, wherein
(i) the VH comprises an amino acid sequence having at least 80%, at least 85%, at least 90%, at least 95%, at least 98%, at least 99%, or 100% sequence identity to SEQ ID NO: 1 and the VL comprises an amino acid sequence having at least 80%, at least 85%, at least 90%, at least 95%, at least 98%, at least 99%, or 100% sequence identity to SEQ ID NO: 2; or
(ii) the VH comprises an amino acid sequence having at least 80%, at least 85%, at least 90%, at least 95%, at least 98%, at least 99%, or 100% sequence identity to SEQ ID NO: 3 and the VL comprises an amino acid sequence having at least 80%, at least 85%, at least 90%, at least 95%, at least 98%, at least 99%, or 100% sequence identity to SEQ ID NO: 4; or
(iii) the VH comprises an amino acid sequence having at least 80%, at least 85%, at least 90%, at least 95%, at least 98%, at least 99%, or 100% sequence identity to SEQ ID NO: 35 and the VL comprises an amino acid sequence having at least 80%, at least 85%, at least 90%, at least 95%, at least 98%, at least 99%, or 100% sequence identity to SEQ ID NO: 36; or
(iv) the VH comprises an amino acid sequence having at least 80%, at least 85%, at least 90%, at least 95%, at least 98%, at least 99%, or 100% sequence identity to SEQ ID NO: 37 and the VL comprises an amino acid sequence having at least 80%, at least 85%, at least 90%, at least 95%, at least 98%, at least 99%, or 100% sequence identity to SEQ ID NO: 38.

16. The antibody or the antigen binding fragment thereof of claim 14, wherein: (a) the antibody is of an isotype selected from the group consisting of IgG, IgA, IgM, IgE and IgD; (b) the antibody is of an subtype selected from the group consisting of IgG1, IgG2, IgG3, and IgG4; (c) the antibody comprises a heavy chain constant region comprising an amino acid sequence having at least 80%, at least 85%, at least 90%, at least 95%, at least 98%, at least 99%, or 100% sequence identity to SEQ ID NO: 19 or 21, and/or a light chain constant region comprising an amino acid sequence having at least 80%, at least 85%, at least 90%, at least 95%, at least 98%, at least 99%, or 100% sequence identity to SEQ ID NO: 18; (d) the antigen binding fragment is selected from the group consisting of Fab, Fab′, F(ab′)2, Fd, Fd′, Fv, scFv, ds-scFv and dAb; (e) the antibody is a murine, human, or humanized antibody; and/or (f) the antibody is a monoclonal antibody.

17. (canceled)

18. (canceled)

19. (canceled)

20. (canceled)

21. (canceled)

22. The antibody or the antigen binding fragment thereof of claim 16, wherein the antibody comprises a heavy chain comprising an amino acid sequence having at least 80%, at least 85%, at least 90%, at least 95%, at least 98%, at least 99%, or 100% sequence identity to SEQ ID NO: 40 and a light chain comprising an amino acid sequence having at least 80%, at least 85%, at least 90%, at least 95%, at least 98%, at least 99%, or 100% sequence identity to SEQ ID NO: 41.

23. The antibody or the antigen binding fragment thereof of claim 14, wherein the antibody is a bi-specific or a multi-specific antibody.

24. The antibody or the antigen binding fragment thereof of claim 23, wherein the antibody is a bispecific antibody which further comprises a second antigen binding region binding to a second antigen and/or wherein the second antigen is a tumor associated antigen or an immune cell antigen, e.g., a T-cell antigen.

25. (canceled)

26. A nucleic acid comprising a nucleotide sequence encoding the antibody or the antigen binding fragment thereof according claim 14; optionally in a vector.

27. (canceled)

28. (canceled)

29. A pharmaceutical composition comprising (i) the antibody or the antigen binding fragment thereof according to claim 14; and (ii) a pharmaceutically acceptable carrier or adjuvant.

30. (canceled)

31. A method of treating a disease in a subject, comprising administering to the subject an effective amount of the antibody or the antigen binding fragment thereof according to claim 14.

32. The method of claim 31, wherein the disease is a cancer (e.g., hepatocellular carcinoma) or a disease or condition caused by viral infection (e.g., a disease or condition caused by HIV infection); optionally, further comprising administering to the subject a second therapeutic agent, optionally, wherein the second therapeutic agent is selected from an antibody, a chemotherapeutic agent and a small molecule drug.

33. (canceled)

34. (canceled)


SEQUENCE LISTING (41 sequences)

SEQ ID NO: 1 - protein, 112 aa, synthetic construct
  REGION 1..112: note = CH101 VH amino acid sequence
EVQLEESGPDLVKPSQSLSLTCTVTGYSITSGFAWHWLRQFPGNRLEWMGYIHYSGSTNYNPSLKSRISITRDTSKNQFFLQLNSVTTEDTATFYCARSDFWGQGTFVTVSS

SEQ ID NO: 2 - protein, 117 aa, synthetic construct
  REGION 1..117: note = CH101 VK amino acid sequence
DVVMTQTPLNLSVTFGQPVSISCTSSQSLLDRDGETFMNWLFQRPGQSPKRLIYLVSKLDFGVPDRFTGSGSGTDFTLKISRVEAEDLGIYYCWQGTHFPLTFGGGTKLEIKRGQAG

SEQ ID NO: 3 - protein, 112 aa, synthetic construct
  REGION 1..112: note = hufrCH101 VH amino acid sequence
QVQLQESGPGLVKPSETLSLTCTVSGYSITSGFAWHWIRQPPGKGLEWIGYIHYSGSTNYNPSLKSRVTISVDTSKNQFSLKLSSVTAADTAVYYCARSDFWGQGTLVTVSS

SEQ ID NO: 4 - protein, 117 aa, synthetic construct
  REGION 1..117: note = hufrCH101 VK amino acid sequence
DVVMTQSPLSLPVTLGQPASISCTSSQSLLDRDGETFMNWFQQRPGQSPRRLIYLVSKLDFGVPDRFSGSGSGTDFTLKISRVEAEDVGVYYCWQGTHFPLTFGGGTKVEIKRGQAG

SEQ ID NO: 5 - DNA, 336 bp, synthetic construct
  misc_feature 1..336: note = CH101 VH nucleic acid sequence
  source 1..336: mol_type = other DNA
gaggtgcagctcgaggagtcaggacctgacctggtgaaaccttctcagtcactttcactcacctgcactgtcactggctattccatcaccagtggttttgcctggcactggctccggcagtttccaggaaacagactggaatggatgggctacatacactatagtggaagcactaactacaacccatctctcaaaagtcgaatctctatcactcgagacacatccaagaatcagttcttcctgcagttgaattctgtgactactgaggacacagccactttttactgtgcaaggtctgacttctggggccaaggcacctttgtcacagtctcctca

SEQ ID NO: 6 - DNA, 351 bp, synthetic construct
  misc_feature 1..351: note = CH101 VK nucleic acid sequence
  source 1..351: mol_type = other DNA
gatgttgtgatgacccagactccactcaatttgtcggtcacctttggacaaccagtctccatctcttgcacgtctagtcagagcctcttagatcgtgatggagagacattcatgaattggttgttccagcggccaggccagtctccaaagcgcctaatctatctggtgtctaaactggactttggagtccctgacaggttcactggcagtggatcagggacagatttcacactgaaaatcagcagagtggaggctgaggatttgggaatttattattgctggcaaggtacacattttcctctcacgttcggtggtgggaccaagctggaaataaaacgcggccaggccggc

SEQ ID NO: 7 - DNA, 336 bp, synthetic construct
  misc_feature 1..336: note = hufrCH101 VH nucleic acid sequence
  source 1..336: mol_type = other DNA
caggttcagctccaggagtcaggacccggtctggtgaaaccttcagagacactgagtctgacatgtacggtaagtgggtactccattacttccggttttgcttggcattggatacggcaaccgcctgggaaggggttggagtggataggttatattcattatagcgggtcaacaaattataacccatcactcaagtcccgcgtcacgataagcgttgatacctccaagaatcagttcagcttgaaattgtcaagcgtaacagccgccgataccgcagtgtattattgcgcaaggagtgacttttggggccaaggtacgcttgtcaccgtaagttcc

SEQ ID NO: 8 - DNA, 351 bp, synthetic construct
  misc_feature 1..351: note = hufrCH101 VK nucleic acid sequence
  source 1..351: mol_type = other DNA
gacgtagtcatgacccagtccccactgtcattgccggtaacgcttggacagcctgctagtatctcatgcacatccagccaatccctccttgaccgcgacggtgagacgtttatgaattggttccagcagagaccaggacagtcaccgaggagacttatttaccttgtttcaaagctcgacttcggtgtccccgacagatttagcggtagtggttctgggacagatttcacccttaaaatcagtagggtcgaagcggaggacgtgggtgtctactattgttggcaaggcacgcactttccattgacctttggcggggggacaaaggtggaaattaaacggggccaggcgggg

SEQ ID NO: 9 - protein, 99 aa, Homo sapiens
QVQLQESGPGLVKPSETLSLTCTVSGGSVSSGGYYWSWIRQPPGKGLEWIGYIYYSGSTNYNPSLKSRVTISVDTSKNQFSLKLSSVTAADTAVYYCAR

SEQ ID NO: 10 - protein, 101 aa, Homo sapiens
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPP

SEQ ID NO: 11 - protein, 15 aa, Homo sapiens
YFDYWGQGTLVTVSS

SEQ ID NO: 12 - protein, 11 aa, Homo sapiens
TFGGGTKVEIK

SEQ ID NO: 13 - DNA, 297 bp, Homo sapiens
  source 1..297: mol_type = genomic DNA
caggtgcagctgcaggagagcggccccggcctggtgaagcccagcgagaccctgagcctgacctgcaccgtgagcggcggcagcgtgagcagcggcggctactactggagctggatcaggcagccccccggcaagggcctggagtggatcggctacatctactacagcggcagcaccaactacaaccccagcctgaagagcagggtgaccatcagcgtggacaccagcaagaaccagttcagcctgaagctgagcagcgtgaccgccgccgacaccgccgtgtactactgcgccagg

SEQ ID NO: 14 - DNA, 303 bp, Homo sapiens
  source 1..303: mol_type = genomic DNA
gacgtggtgatgacccagagccccctgagcctgcccgtgaccctgggccagcccgccagcatcagctgcaggagcagccagagcctggtgtacagcgacggcaacacctacctgaactggttccagcagaggcccggccagagccccaggaggctgatctacaaggtgagcaacagggacagcggcgtgcccgacaggttcagcggcagcggcagcggcaccgacttcaccctgaagatcagcagggtggaggccgaggacgtgggcgtgtactactgcatgcagggcacccactggcccccc

SEQ ID NO: 15 - DNA, 45 bp, Homo sapiens
  source 1..45: mol_type = genomic DNA
tactttgactactggggccaaggaaccctggtcaccgtctcctca

SEQ ID NO: 16 - DNA, 33 bp, Homo sapiens
  source 1..33: mol_type = genomic DNA
actttcggcggagggaccaaggtggagatcaaa

SEQ ID NO: 17 - DNA, 321 bp, Homo sapiens
  source 1..321: mol_type = genomic DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 18 - protein, 107 aa, Homo sapiens [sequence omitted]

SEQ ID NO: 19 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 990 bp, Homo sapiens
  source 1..990: mol_type = genomic DNA
gcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 21 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - DNA, 981 bp, Homo sapiens
  source 1..981: mol_type = genomic DNA
gcgtcgaccaagggaccaagcgtattcccgcttgcgccatgtagcagatcaacaagtgagtccacggcggcccttgggtgcttggtaaaggattactttcctgagccagtaaccgtgtcttggaattcaggagctctcacgtcaggggtacacaccttccctgccgtccttcaatcttctggcctctatagcctctcaagcgtcgttactgtgccatcctcctcactcggaactaaaacatatacttgcaacgtagaccacaagccctcaaatacaaaagttgacaaacgagttgaatctaagtacggcccgccatgtccgccatgtccagcccccgaattcttggggggaccatccgtatttcttttcccgccgaaacccaaagacaccttgatgataagccgaacaccagaggttacatgtgtcgttgttgatgtctctcaggaagatcctgaggtccagtttaactggtacgtagatggggtggaagttcacaacgcaaaaaccaagccgagagaggaacagttcaactcaacctatcgggttgtttccgttctcacagttctgcatcaggactggcttaatggaaaagaatacaagtgtaaagtgtcaaataagggcctcccatcaagcattgaaaagaccatatctaaggccaaaggccaacctcgcgagcctcaagtctacaccttgcctcctagccaagaagaaatgacaaagaaccaagtatctctcacatgtcttgttaaagggttctatccaagcgatatcgctgttgagtgggaaagcaatggccaacctgaaaataactacaaaaccactcctcccgtgcttgactcagatggttctttcttcctctattcacgattgactgtggacaaaagtcgctggcaggaagggaacgttttctcctgtagcgtgatgcacgaggcgctccacaatcattatacccaaaagtcactctctcttagccttggcaag

SEQ ID NO: 23 - protein, 8 aa, synthetic construct
  REGION 1..8: note = CH101 VH CDR1
GYSITSGF

SEQ ID NO: 24 - protein, 5 aa, synthetic construct
  REGION 1..5: note = CH101 VH CDR2
HYSGS

SEQ ID NO: 25 - skipped

SEQ ID NO: 26 - protein, 16 aa, synthetic construct
  REGION 1..16: note = CH101 VL CDR1
TSSQSLLDRDGETFMN

SEQ ID NO: 27 - protein, 7 aa, synthetic construct
  REGION 1..7: note = CH101 VL CDR2
LVSKLDF

SEQ ID NO: 28 - protein, 8 aa, synthetic construct
  REGION 1..8: note = CH101 VH CDR3
WQGTHFPL

SEQ ID NO: 29 - protein, 8 aa, synthetic construct
  REGION 1..8: note = CH34 VH CDR1
GFTFSDYG

SEQ ID NO: 30 - protein, 8 aa, synthetic construct
  REGION 1..8: note = CH34 VH CDR2
ISSLAYTI

SEQ ID NO: 31 - protein, 9 aa, synthetic construct
  REGION 1..9: note = CH34 VH CDR3
ARGGAGFAY

SEQ ID NO: 32 - protein, 6 aa, synthetic construct
  REGION 1..6: note = CH34 VL CDR1
DHINNW

SEQ ID NO: 33 - skipped

SEQ ID NO: 34 - protein, 9 aa, synthetic construct
  REGION 1..9: note = CH34 VL CDR3
QQYWSTPWT

SEQ ID NO: 35 - protein, 115 aa, synthetic construct
  REGION 1..115: note = Mouse CH34 VH
EVKLVESGGGLVQPGGSRKLSCAASGFTFSDYGMAWVRQAPGKGPEWVAFISSLAYTIYYADTVTGRFTISRENAKNTLYLEMSSLRSEDTAMYYCARGGAGFAYWGQGTLVTVS

SEQ ID NO: 36 - protein, 107 aa, synthetic construct
  REGION 1..107: note = Mouse CH34 VL
DIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIK

SEQ ID NO: 37 - protein, 115 aa, synthetic construct
  REGION 1..115: note = Humanized CH34 VH
EVQLVESGGGLVKPGGSLRLSCAASGFTFSDYGMSWIRQAPGKGLEWVSYISSLAYTIYYADSVKGRFTISRDNAKNSLYLQMNSLRAEDTAVYYCARGGAGFAYWGQGTLVTVS

SEQ ID NO: 38 - protein, 107 aa, synthetic construct
  REGION 1..107: note = Humanized CH34 VL
DIQMTQSPSSLSASVGDRVTITCRASDHINNWLAWYQQKPGKAPKLLLYGATRLESGVPSRFSGSGSGTDYTLTISSLQPEDFATYYCQQYWSTPWTFGGGTKVEIK

SEQ ID NO: 39 - protein, 19 aa, synthetic construct
  REGION 1..19: note = Signal peptide
MGWSCIILFLVATATGVHS

SEQ ID NO: 40 - protein, 452 aa, synthetic construct
  REGION 1..452: note = Mouse CH34 heavy chain
EVKLVESGGGLVQPGGSRKLSCAASGFTFSDYGMAWVRQAPGKGPEWVAFISSLAYTIYYADTVTGRFTISRENAKNTLYLEMSSLRSEDTAMYYCARGGAGFAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 41 - protein, 214 aa, synthetic construct
  REGION 1..214: note = Mouse CH34 kappaK chain
DIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC